(19) United States
(12) Patent Application Publication
(10) Pub. No.: US 2009/0054507 A1
(43) Pub. Date: Feb. 26, 2009

(54) CONTROL OF MALIGNANT CELLS BY KINASE INHIBITION

(75) Inventor: Michael Kalafatis, Shaker Heights, OH (US)

Correspondence Address: RANKIN, HILL & CLARK LLP, 38210 Glenn Avenue, WILLOUGHBY, OH, 44094-7808, US

(73) Assignee: CLEVELAND STATE UNIVERSITY, Cleveland, OH (US)

(21) Appl. No.: 12/201,693

(22) Filed: Aug. 29, 2008

Related U.S. Application Data
(63) Continuation-in-part of application No. PCT/US2007/019676, filed on Sep. 11, 2007.
(60) Provisional application No. 60/844,022, filed on Sep. 12, 2006.
(60) Provisional application No. 60/969,064, filed on Aug. 30, 2007.
(60) Provisional application No. 60/969,184, filed on Aug. 31, 2007.

(51) Int. Cl. A61K 31/4184; A61K 31/4192

(52) U.S. Cl. 514/395; 514/359

(57) ABSTRACT

Inhibitors of casein kinase 2 are described that have been found to arrest uncontrolled cell proliferation, thereby suggesting their use in cancer treatment strategies. Specific applications include treating breast cancer, colon cancer, melanoma, chronic myelogenous leukemia, bladder cancer, renal cancer, and brain cancer. Various methods and compositions utilizing the inhibitors are described.

### CROSS REFERENCE TO RELATED APPLICATIONS

This application is a continuation-in-part (CIP) of international application No. PCT/US2007/019676 filed Sep. 11, 2007, which claims priority upon U.S. provisional application Ser. No. 60/844,022 filed Sep. 12, 2006. This application also claims priority upon U.S. provisional application Ser. No. 60/969,064 filed Aug. 30, 2007. This application also claims priority upon U.S. provisional application Ser. No. 60/969,184 filed Aug. 31, 2007.

### STATEMENT REGARDING FEDERALLY SPONSORED RESEARCH OR DEVELOPMENT

This work was supported by grant HL-071625 from the National Heart Lung and Blood Institutes.

### FIELD OF THE INVENTION

The presently disclosed embodiments relate to the control of malignant cells by inhibiting certain kinases. More specifically, the embodiments are directed to methods and compositions involving the use of particular casein kinase 2 inhibitors in the treatment of cancer.

### BACKGROUND OF THE INVENTION

Cancer cells are characterized by increased proliferation and loss of the cells' normal phenotype and function. Many types of cancer are caused by defects in signaling pathways including deregulation of a process known as apoptosis. Apoptosis is a genetically programmed and evolutionary conserved mechanism through which the normal development and tissue homeostasis are maintained.

Cancer development generally requires that tumor cells achieve certain characteristics, including increased replicative potentials, anchorage and growth-factor independency, departure from apoptosis, angiogenesis and metastasis. Many of these processes involve the actions of protein kinases, which have emerged as key regulators of many aspects of abnormal and uncontrolled cell growth. Disrupted protein kinase activity is repeatedly found to be associated with human malignancies, making these proteins attractive targets for anti-cancer therapy.

The reciprocal chromosomal translocation t(9;22), known as the Philadelphia positive chromosome (Ph+) is associated with diseases like chronic myelogenous leukemia (CML), acute myelogenous leukemia (AML), acute non-lymphocytic leukemia (ANLL) and acute lymphocytic leukemia (ALL). This genetic abnormality results in the chimeric oncoprotein BCR/ABL tyrosine kinase, which is thought to be the main cause of the abnormal survival and over-proliferation of hematopoietic stem cells and their progeny. In chronic myelogenous leukemia, the BCR/ABL tyrosine kinase is constitutively activated. Different intracellular pathways are transformed by the oncoprotein BCR/ABL, resulting in uncontrolled hematopoietic proliferation.

The late phase of chronic myelogenous leukemia, named blast crisis (or blastic phase), is characterized by extreme overproliferation of stem cells and their progeny in bone marrow. In blast crisis, a major complication is thrombosis due to high platelet counts. In myeloproliferative disorders, like chronic myelogenous leukemia, the platelet counts and function are abnormal due to overproliferation of malignant megakaryoblasts.

In different cancers, including leukemias, a tyrosine kinase named casein kinase 2 (CK2) was found to be constitutively activated, elevated and serving as an oncoprotein. CK2 is a pleiotropic, ubiquitous Ser/Thr kinase. The protein is a heterotetramer with two catalytic subunits, α and α′, and two regulatory β subunits. Each subunit was shown to be able to execute specific functions by itself or in the holoenzyme form, the αα′β2 tetramer. The up regulation and hyperactivity of CK2 has an anti-apoptotic effect which is associated with decreased platelet counts and function in leukemias, such as acute myelogenous leukemia and chronic myelogenous leukemia. Interestingly, CK2α was found to be a substrate for the ABL domain of BCR/ABL and forms a specific complex with the BCR domain of BCR/ABL. It was hypothesized that CK2α sterically impedes the binding of the ABL SH2 domain to BCR. This results in proliferation abnormalities in Philadelphia positive cells. Therefore CK2α was shown to be a possible arbitrator of BCR/ABL function. Other functions of CK2α downstream of the BCR/ABL interaction yield an overall oncogenic response in Philadelphia positive cells.

CK2α protein kinase inhibitors have been developed and studied, such as Emodin; 5,6-dichloro-1-beta-D-ribofuranosylbenzimidazole (DRB); 4,5,6,7-Tetrabromobenzotriazole (TBB); 2-Dimethylamino-4,5,6,7-tetrabromo-1H-benzimidazole (DMAT); and ellagic acid. Inhibition of CK2 in various cancer cell lines produced apoptosis and proliferation arrest.

Although it is believed that CK2 can serve an anti-apoptic role by protecting regulatory proteins from caspase-mediated degradation, the exact mechanisms are not well understood. Furthermore, although protein kinase activity has been linked with many forms of human cancers, specific treatment methodologies using CK2 protein kinases are still needed. Accordingly, a need exists for a strategy by which abnormal cell proliferation can be arrested by controlling CK2 protein kinases. More desirably, it would be beneficial to identify a method of inducing arrest of cell proliferation using CK2 protein kinases while maintaining steady cell numbers. And, it would also be beneficial to provide such a method without attendant problems of cell necrosis.

### SUMMARY OF THE INVENTION

The difficulties and drawbacks associated with previous methodologies are overcome in the present methods and compositions relating to the use of particular CK2α inhibitors. The particular inhibitors and their administration have been discovered to induce arrest of cell proliferation while maintaining steady cell numbers, and particularly without necrosis. Moreover, the particular inhibitors and their administration have been discovered to arrest over-proliferation of certain cells.

In one aspect, the present invention provides a method for treating a disease characterized by over-proliferation of malignant cells. Examples of such diseases are (i) breast cancer, (ii) colon cancer, (iii) skin cancer, (iv) chronic myelogenous leukemia, (v) renal cell carcinoma, (vi) bladder cancer, and (vii) glioblastoma. The method comprises selectively inhibiting CK2α activity.

In another aspect, the present invention provides a method for treating a disease characterized by over-proliferation of malignant cells. Examples of such diseases include (i) breast cancer, (ii) colon cancer, (iii) skin cancer, (iv) chronic myelogenous leukemia, v) renal cell carcinoma, (vi) bladder cancer, and (vii) glioblastoma. The method comprises administering an effective amount of a CK2α inhibitor to a patient in need of treatment.

And in yet another aspect, the present invention provides a pharmaceutical composition comprising a CK2α selective inhibitor selected from the group consisting of (i) 4,5,6,7-Tetrabromobenzotriazole (TBBt), (ii) 2-Dimethylamino-4,5,6,7-tetrabromo-1H-benzimidazole (DMAT), and combinations of (i) and (ii). The pharmaceutical composition also comprises a pharmaceutically acceptable carrier.

As will be realized, the invention is capable of other and different embodiments and its several details are capable of modifications in various respects, all without departing from the invention. Accordingly, the drawings and description are to be regarded as illustrative and not restrictive.

### BRIEF DESCRIPTION OF THE DRAWINGS

FIG. 1 includes photographs of MEG-01 cells prior to and after treatment with a preferred embodiment inhibitor.

FIG. 2 includes photographs of MEG-01 cells undergoing thrombocytopoiesis, induced by a preferred inhibitor.

FIGS. 3-5 are photographs of MEG-01 cells producing platelets after inducement with a preferred inhibitor.

FIGS. 6-7 are photographs of activated platelet-like particles from MEG-01 cells, the cells having been treated with a preferred inhibitor.

FIG. 8 is a graph further illustrating the effect of preferred embodiment inhibitors upon MEG-01 cells.

FIG. 9 is a photograph of a control untreated colony of MEG-01 cells.

FIG. 10 is a photograph of a colony of MEG-01 cells treated with a preferred embodiment inhibitor.

FIG. 11 is a graph comparing colony areas of a control to a sample treated in accordance with a preferred embodiment inhibitor.

FIG. 12 is a graph of a DNA content assay referring to the preferred embodiment inhibitors against a control.

FIG. 13 is a graph further illustrating the preferred embodiment inhibitors against a control.

FIG. 14 is a DNA content analysis of MEG-01 cell lines treated with a preferred embodiment inhibitor.

FIGS. 15 and 16 are graphs showing apoptosis and phenotype change of a control and MEG-01 cell lines treated with a preferred inhibitor, respectively.

FIG. 17 is a graph illustrating the effect of preferred inhibitors on MEG-01 cell lines after 24 hours.

FIG. 18 is a graph illustrating the effect of preferred inhibitors on MEG-01 cell lines after 48 hours.

FIG. 19 is a graph illustrating the effect of preferred inhibitors on MEG-01 cell lines after 72 hours.

FIG. 20 is a graph illustrating the effect of preferred inhibitors on MEG-01 cell lines after 96 hours.

FIG. 21 is a photograph of untreated control MEG-01 cells after 96 hours.

FIG. 22 is a photograph of cells treated with a preferred inhibitor after 96 hours.

FIG. 23 is a photograph of proplatelets formation following treatment with a preferred inhibitor between 72 and 96 hours.

FIG. 24 is a SEM micrograph of MEG-01 cells.

FIG. 25 is a photograph of proplatelets formation following treatment with a preferred embodiment inhibitor.

FIG. 26 is a photograph of proplatelets bearing MEG-01 megakaryocytes following treatment with a preferred inhibitor after 72 to 96 hours.

FIG. 27 is a photograph of platelet-like particles following treatment with a preferred inhibitor after 72 to 96 hours.

FIG. 28 is a graph illustrating activation of platelets from MEG-01 cells obtained by use of a preferred inhibitor, compared to a control.

FIG. 29 is a graph illustrating activation of platelets from MEG-01 cells obtained by use of a preferred inhibitor, compared to a control.

FIG. 30 is a graph illustrating activation of platelets from MEG-01 cells obtained by use of a preferred inhibitor, compared to a control.

FIG. 31 is a graph illustrating activation of platelets from MEG-01 cells obtained by use of a preferred inhibitor, compared to a control.

FIG. 32 is a photograph of a fibrin clot formed from platelets derived from use of a preferred inhibitor.

FIG. 33 is another photograph of a fibrin clot formed from platelets derived from use of a preferred inhibitor.

FIG. 34 is yet another photograph of a fibrin clot formed from platelets derived from use of a preferred inhibitor.

FIG. 35 is a graph illustrating changes in tumor volume of MEG-01 cells treated with a preferred inhibitor compared to a control.

FIG. 36 is a graph illustrating changes in tumor volume of MEG-01 cells treated with a preferred inhibitor compared to a control.

FIG. 37 is a graph of platelet counts of a MEG-01 xenograft treated with a preferred inhibitor as compared to a control.

FIG. 38 is a graph of percentage abnormal cells of a control, normal mice, and an inhibitor-treated xenograph.

FIG. 39 is a graph of tail bleeding times in an inhibitor-treated MEG-01 xenograft mice compared to a control and normal mice.

FIG. 40 is a graph of spleen size in an inhibitor-treated MEG-01 xenograft mice compared to a control and normal mice.

FIG. 41 is a graph of apoptotic-necrotic areas in MEG-01 cells and a control.

FIG. 42 is a graph of angiogenesis areas in MEG-01 cells and a control.

FIG. 43 is a graph of cell counts in vitro of MCF-7 cells and a control.

FIGS. 44-49 are photographs of MCF-7 cells (controls and inhibitor treated) after 24 hours.

FIGS. 50-52 are graphs showing apoptosis and phenotype change in a control and MCF-7 cell lines treated with a preferred inhibitor.

FIG. 53 is a graph comparing percentages of apoptotic cells in the samples depicted in FIGS. 50-52.

FIGS. 54-56 are photographs and a graph illustrating anchorage independence of a MCF-7 cell line.

FIG. 57 is a graph illustrating changes in tumor size in MCF-7 cells and a control.

FIG. 58 is a graph of apoptotic-necrotic areas in MCF-7 cells and a control.

FIG. 59 is a graph of angiogenesis areas in MCF-7 cells and a control.

FIG. 60 is a graph of cell counts in vitro of SW-480 cells and a control.

FIGS. 61-66 are photographs of SW-480 cells (controls and inhibitor treated) after 24 hours.

FIGS. 67-69 are graphs showing apoptosis and phenotype change in a control and SW-480 cell lines treated with a preferred inhibitor.

FIG. 70 is a graph comparing percentages of apoptotic cells in the samples depicted in FIGS. 67-69.

FIGS. 71-73 are photographs and a graph illustrating anchorage independence of a SW-480 cell line.

FIG. 74 is a graph illustrating changes in tumor size in SW-480 cells and a control.

FIG. 75 is a graph of apoptotic-necrotic areas in SW-480 cells and a control.

FIG. 76 is a graph of angiogenesis areas in SW-480 cells and a control.

FIG. 77 is a graph of cell counts in vitro of WM-164 cells and a control.

FIGS. 78-83 are photographs of WM-164 cells (controls and inhibitor treated) after 24 hours.

FIGS. 84-86 are graphs showing apoptosis and phenotype change in a control and WM-164 cell lines treated with a preferred inhibitor.

FIG. 87 is a graph comparing percentages of apoptotic cells in the samples shown in FIGS. 84-86.

FIGS. 88-90 are photographs and a graph illustrating anchorage independence of a WM-164 cell line.

FIG. 91 is a graph illustrating changes in tumor size in WM-164 cells and a control.

FIG. 92 is a graph of apoptotic-necrotic areas in WM-164 cells and a control.

FIG. 93 is a graph of angiogenesis areas in WM-164 cells and a control.

FIG. 94 is a graph illustrating changes in tumor size in another cell line, ACHN.

FIG. 95 is a graph illustrating changes in tumor size in another cell line, HT1376.

FIG. 96 is a graph illustrating changes in tumor size in another cell line, U87.

### DETAILED DESCRIPTION OF THE EMBODIMENTS

Cancer is so widespread and lethal that it can be considered the biggest health problem of this century. Various unknown causes, diverse genetic and protein abnormalities, as well as different and complex molecular mechanisms make cancer drug development a real challenge. Harmful side-effects are another problem that needs to be overcome in the search for possible cancer therapies. However, while the reason for developing cancer may be different from person to person, the end point is the same: cells are growing abnormally and uncontrollably. Casein kinase 2 (CK2) was found to be up-regulated and over-expressed in tumor tissue and may be responsible for cancer growth and sustainability. Data presented herein using various CK2 inhibitors (antisense nucleotides or ATP analogues) in malignant cell lines as well as in murine xenografts demonstrated that the cells undergo apoptosis and proliferation arrest in vitro and in vivo. Thus, such inhibitors may be potent anti-carcinogenes. The extensive data presented herein obtained with several different malignant cells lines in culture and with mice xenografts demonstrates that inhibition of CK2 that is normally located in the cytoplasm of normal cells, and is translocated into the nucleus of tumor cells, may be a general treatment for all cancers. MEG-01 cells are malignant megakaryoblasts isolated from a patient with chronic myelogenous leukemia in blast crisis. MCF-7 cells are breast cancer cells, estrogen dependent with a highly abnormal proliferation rate. SW-480 cells are malignant colon cells (epithelial cancer cells). WM-164 cells are very aggressive melanoma cells. Such cells are resistant to apoptosis and manifest anchorage independence by growing colonies in soft agar. A specific CK2 inhibitor induced proliferation arrest and apoptosis in all these cell lines when tested in vitro (in cell culture) and in vivo (with mice xenografts). All treated tumors showed necrosis, apoptosis and reduced angiogenesis versus untreated tumors. The liver, brain, kidney, and muscle tissue of all mice treated with the inhibitor appeared to be normal following histological analysis.

Although not wishing to be limited to any particular theory, it is believed that CK2 is localized in the cytoplasm of normal cells; CK2 is translocated in the nucleus of malignant cells and phosphorylates a protein or a group of proteins responsible for normal cell growth. Phosphorylation of these proteins will severely impair their normal function resulting in uncontrollable cell growth. Thus, specific CK2 inhibitors could be used as anti-cancer drugs and will stop abnormal cell proliferation independently of the type of cancer. These inhibitors will have no side effect because they have no effect on normal cells. Thus, CK2 has common targets in the nucleus of malignant cells. Inhibition of phosphorylation of these target proteins can be used as a starting point for the development of a potential cancer therapy.

### Basis for Treatment Strategy

As noted, although not wishing to be bound to any particular theory, it is proposed that CK2 is localized in the cytoplasma of normal cells; CK2 is translocated in the nucleus of malignant cells and phosphorylates a protein or a group of proteins responsible for normal cell growth. Phosphorylation of these proteins will severely impair their normal function resulting in uncontrollable cell growth. Thus, specific CK2 inhibitors could be used as anti-cancer drugs and will stop abnormal cell proliferation independently of the type of cancer. These inhibitors have no side effect because they have no effect on normal cells.

An important conclusion from the findings presented herein is that the mechanism by which CK2 stops abnormal cell proliferation is similar in each cell line.

### Subunits of the Protein Kinase CK2

In many organisms, distinct isoenzymic forms of the catalytic subunit of CK2 have been identified. For example, in humans, two catalytic isoforms, designated CK2α and CK2α′, have been well characterized, while a third isoform, designated CK2α″, has been identified recently. In humans, only a single regulatory subunit, designated CK2β, has been identified, but multiple forms of CK2β have been identified in other organisms.

At a very early stage after its discovery, CK2, together with a distinct casein kinase designed ‘casein kinase I’ (now known as protein kinase CK1), was distinguished among known protein kinases for its ability to phosphorylate serine or threonine residues. In their domains, CK2α and CK2α′ exhibit approximately 90% identity which is consistent with the fact that they display similar enzymic properties (including turnover rates and substrate specificity) in vitro. In contrast with the high similarity that is seen within their catalytic domains, the C-terminal domains of CK2α and CK2α′ are completely unrelated. Very little is currently known about CK2α″, which was identified only recently.

Given the complex nature of CK2, in terms of its large number of potential substrates and its participation in a broad array of cellular processes, it is inevitable that many more isoform-specific functions or interactions for each of the CK2 isoforms remain to be defined. To date, much of the literature involving CK2 has not made a distinction between the different isoenzymic forms of CK2. In particular, given the close similarity in the enzymic characteristics of CK2α and CK2α′ (and presumably CK2α″), it is not possible from simple CK2 phosphorylation assays to determine which isoforms are actually contributing to the activity under investigation.

### Thrombocytopoiesis

Megakaryocytes are polyploid cells, originating from hematopoietic stem cells in the bone marrow. Thrombocytopoiesis refers to the production of blood platelets or thrombocytes. More specifically, thrombocytopoiesis is the process of producing of anucleated cells or platelets, from megakaryocytes. Megakaryoblasts are precursors of platelets that first differentiate to the stage of megakaryocytes. Mature megakaryocytes form pseudopodia and give rise to platelets. More specifically, megakaryoblasts undergo endomitosis and maturation to the stage of megakaryocytes, through a process called megakaryocytopoiesis. Proplatelets bearing megakaryocytes fragment to give rise to platelets, through the process of thrombocytopoiesis. Platelets (thrombocytes) are vital for maintaining normal hemostasis and for the response of the body to trauma.

The process of platelet formation is complex and at present, not well understood. The thrombocytopoiesis process has been linked to the constitutive apoptosis of megakaryocytes. Caspase activation in megakaryocytes has also been connected with platelets production. Pro-apoptotic and pro-survival balance are shifted towards apoptosis during megakaryocytopoiesis and thrombocytopoiesis. Isolation and characterization of CK2 from platelets has been achieved and more recently, CK2 has been cloned and sequenced from human platelets and human MEG-01 cells. The present invention is based upon developments undertaken to identify the effect of CK2 and specifically CK2α, on MEG-01 proliferation and subsequent thrombocytopoiesis processes.

MEG-01 cells were previously isolated from a patient with CML, Ph+, in blast crisis, with high peripheral blast counts and thrombocytosis (high platelets counts). The cells were characterized as being megakaryoblasts in an early stage of differentiation in the megakaryocytic lineage. The cells expressed the integrin α_{IIb}β₃ on their surface and were positive for platelet peroxidase. MEG-01 cells expressed the p210 BCR/ABL tyrosine kinase. MEG-01 cells were found to be cytokine independent and capable of differentiating in vitro in response to PMA, nitric oxide (NO), aphidicolin, nocodazole and staurosporine. MEG-01 cells were found to release platelet-like particles following all of these treatments. Inhibition of caspases in a MEG-01 cell line was shown to result in impaired proplatelet formation and platelets release.

The effect of casein kinase 2 alpha subunit (CK2α) inhibition with specific preferred embodiment inhibitors was studied in a megakaryoblastic cell line from a CML patient in blast crisis (MEG-01). It was surprisingly discovered that the preferred embodiment casein kinase 2 inhibitors induce proliferation arrest while maintaining a steady cell number for an extended time period, such as one week. Treated cells grew at a significantly lower rate than non-treated cells. Apoptosis of MEG-01 was induced by the preferred embodiment CK2 inhibitors, and the apoptosis was dose and time dependent. No necrosis was detected in the presence of the inhibitors, demonstrating that the preferred compounds are not cytotoxic. In the presence of the preferred embodiment CK2 inhibitors, megakaryocytes matured to a pro-platelets bearing stage. Platelets were subsequently released through rupture, following cytoplasmic fragmentation and nuclear extrusion. Thrombocytopoiesis due to the use of the preferred embodiment CK2 inhibitors occurred both in suspension and with MEG-01 cells grown on a fibronectin matrix. Platelets obtained following these treatments were found to undergo shape change in response to various agonists. The platelets obtained in culture, following CK2α inhibition with specific kinase inhibitors were functional. These platelets formed a clot visible with the eye (a normal fibrin clot as seen by SEM), when exposed to agonists. Thus, by using the preferred embodiment CK2 inhibitors, the abnormal proliferation of a transformed cell line was successfully stopped and its path reversed towards its normal function.

In accordance with the present invention, CK2α inhibition studies with the preferred inhibitors, demonstrate a key role of CK2 in oncogenic development as well as in the megakaryocytopoiesis and thrombocytopoiesis processes. These significant advances suggest a wide array of potential applications and CK2 targeted drug design for patients with cytokine and BCR/ABL inhibitors resistance. Furthermore, due to the importance of protein kinases in malignant processes, the present invention has significant future therapeutic interest.

### The Preferred Inhibitors

In accordance with the present invention, the preferred embodiment CK2α inhibitors are DMAT and TBB. DMAT is 2-dimethylamino-4,5,6,7-tetrabromo-1H-benzimidazole and has the following structural formula (1):

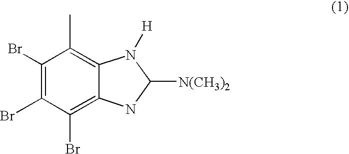

TBB (or sometimes referred to as TBBt) is 4,5,6,7-tetrabromobenzotriazole and has the following structural formula (2):

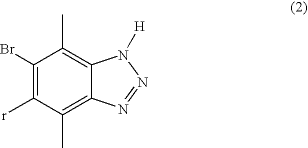

The present invention includes the use of either or both of the inhibitors DMAT and TBB, and/or their pharmaceutically acceptable salts. The preferred inhibitors can be incorporated into a wide array of compositions, formulations, and pharmaceuticals.

### The Preferred Pharmaceutical Compositions

The pharmaceutical compositions may include an inhibitor by itself, or in combination and optionally including one or more suitable diluents, fillers, salts, disintegrants, binders, lubricants, glidants, wetting agents, controlled release matrices, colorants/flavoring, carriers, excipients, buffers, stabilizers, solubilizers, other materials well known in the art and combinations thereof.

Any pharmaceutically acceptable (i.e., sterile and non-toxic) liquid, semisolid, or solid diluents that serve as pharmaceutical vehicles, excipients, or media may be used. Exemplary diluents include, but are not limited to, polyoxyethylene sorbitan monolaurate, magnesium stearate, calcium phosphate, mineral oil, cocoa butter, and oil of theobroma, methyl- and propylhydroxybenzoate, talc, alginates, carbohydrates, especially mannitol, alpha-lactose, anhydrous lactose, cellulose, sucrose, dextrose, sorbitol, modified dextrans, gum acacia, and starch. Some commercially available diluents are Fast-Flo, Emdex, STA-Rx 1500, Erncompress and Avicell. Such compositions may influence the physical state, stability, rate of in vivo release, and rate of in vivo clearance of the inhibitor compounds, see, e.g., Remington's Pharmaceutical Sciences, 18th Ed. pp. 1435-1712 (1990).

Pharmaceutically acceptable fillers can include, for example, lactose, microcrystalline cellulose, dicalcium phosphate, tricalcium phosphate, calcium sulfate, dextrose, mannitol, and/or sucrose. Inorganic salts including calcium triphosphate, magnesium carbonate, and sodium chloride may also be used as fillers in the pharmaceutical compositions. Amino acids may be used such as used in a buffer formulation of the pharmaceutical compositions.

Disintegrants may be included in solid dosage formulations of the inhibitors of the present invention. Materials used as disintegrants include but are not limited to starch including the commercial disintegrant based on starch, Explotab. Additional examples include, but are not limited to sodium starch glycolate, Amberlite, sodium carboxymethylcellulose, ultramylopectin, sodium alginate, gelatin, orange' peel, acid carboxymethylcellulose, natural sponge and bentonite may all be used as disintegrants in the pharmaceutical compositions. Other disintegrants include insoluble cationic exchange resins. Powdered gums including powdered gums such as agar, Karaya or tragacanth may be used as disintegrants and as binders. Alginic acid and its sodium salt are also useful as disintegrants.

Binders may be used to hold the composition, formulation, or pharmaceutical together to form a hard tablet and include materials from natural products such as acacia, tragacanth, starch and gelatin. Others include methyl cellulose (MC), ethyl cellulose (EC) and carboxymethyl cellulose (CMC). Polyvinyl pyrrolidone (PVP) and hydroxypropylmethyl cellulose (HPMC) can both be used in alcoholic solutions to facilitate granulation of the therapeutic ingredient.

An antifrictional agent may be included in the composition, formulation, or pharmaceutical to prevent sticking during the formulation process. Lubricants may be used as a layer between the ingredients and the die wall, and these can include but are not limited to; stearic acid including its magnesium and calcium salts, polytetrafluoroethylene (PTFE), liquid paraffin, vegetable oils and waxes. Soluble lubricants may also be used such as sodium lauryl sulfate, magnesium lauryl sulfate, polyethylene glycol of various molecular weights, Carbowax 4000 and 6000.

Glidants that might improve the flow properties of the composition, formulation, or pharmaceutical during formulation and to aid rearrangement during compression might be added. Suitable glidants include starch, talc, pyrogenic silica and hydrated silicoaluminate.

To aid dissolution of the composition, formulation, or pharmaceutical into an aqueous environment, a surfactant might be added as a wetting agent. Natural or synthetic surfactants may be used. Surfactants may include anionic detergents such as sodium lauryl sulfate, dioctyl sodium sulfosuccinate, and dioctyl sodium sulfonate. Cationic detergents such as benzalkonium chloride and benzethonium chloride may be used. Nonionic detergents that can be used in the pharmaceutical formulations include lauromacrogol 400, polyoxyl 40 stearate, polyoxyethylene hydrogenated castor oil 10, 50 and 60, glycerol monostearate, polysorbate 40, 60, 65 and 80, sucrose fatty acid ester, methyl cellulose and carboxymethyl cellulose. These surfactants can be present in the pharmaceutical compositions of the invention either alone or as a mixture in different ratios.

Controlled release formulations may be desirable. The inhibitors of the invention can be incorporated into an inert matrix which permits release by either diffusion or leaching mechanisms, e.g., gums. Slowly degenerating matrices may also be incorporated into the pharmaceutical formulations, e.g., alginates, polysaccharides. Another form of controlled release is a method based on the Oros therapeutic system (Alza Corp.), i.e., the drug is enclosed in a semipermeable membrane which allows water to enter and push the inhibitor compound out through a single small opening due to osmotic effects. Some enteric coatings also have a delayed release effect.

Colorants and flavoring agents may also be included in the pharmaceutical compositions. For example, the inhibitors of the invention may be formulated (such as by liposome or microsphere encapsulation) and then further contained within an edible product, such as a beverage containing colorants and flavoring agents.

The therapeutic agent can also be given in a film coated tablet. Nonenteric materials for use in coating the pharmaceutical compositions include methyl cellulose, ethyl cellulose, hydroxyethyl cellulose, methylhydroxy-ethyl cellulose, hydroxypropyl cellulose, hydroxypropyl-methyl cellulose, sodium carboxy-methyl cellulose, povidone and polyethylene glycols. Enteric materials for use in coating the pharmaceutical compositions include esters of phthalic acid. A mix of materials might be used to provide the optimum film coating. Film coating manufacturing may be carried out in a pan coater, in a fluidized bed, or by compression coating.

The compositions can be administered in solid, semi-solid, liquid or gaseous form, or may be in dried powder, such as lyophilized form. The pharmaceutical compositions can be packaged in forms convenient for delivery, including, for example, capsules, sachets, cachets, gelatins, papers, tablets, capsules, suppositories, pellets, pills, troches, lozenges or other forms known in the art. The type of packaging will generally depend on the desired route of administration. Implantable sustained release formulations are also contemplated, as are transdermal formulations.

### The Preferred Methods of Treatment

In the preferred treatment methods according to the invention, the inhibitor compounds may be administered by various routes. For example, pharmaceutical compositions may be for injection, or for oral, nasal, transdermal or other forms of administration, including, e.g., by intravenous, intradermal, intramuscular, intramammary, intraperitoneal, intrathecal, intraocular, retrobulbar, intrapulmonary (e.g., aerosolized drugs) or subcutaneous injection (including depot administration for long term release e.g., embedded under the splenic capsule, brain, or in the cornea); by sublingual, anal, vaginal, or by surgical implantation, e.g., embedded under the splenic capsule, brain, or in the cornea. The treatment may consist of a single dose or a plurality of doses over a period of time. In general, the methods of the invention involve administering effective amounts of an inhibitor of the invention together with pharmaceutically acceptable diluents, preservatives, solubilizers, emulsifiers, adjuvants and/or carriers, as described above.

In one aspect, the invention provides methods for oral administration of a pharmaceutical composition of the invention. Oral solid dosage forms are described generally in Remington's Pharmaceutical Sciences, supra at Chapter 89. Solid dosage forms include tablets, capsules, pills, troches or lozenges, and cachets or pellets. Also, liposomal or proteinoid encapsulation may be used to formulate the compositions as for example, proteinoid microspheres reported in U.S. Pat. No. 4,925,673. Liposomal encapsulation may include liposomes that are derivatized with various polymers, e.g., U.S. Pat. No. 5,013,556. In general, the formulation will include a compound of the invention and inert ingredients which protect against degradation in the stomach and which permit release of the biologically active material in the intestine.

The inhibitors can be included in the formulation as fine multiparticulates in the form of granules or pellets of particle size about 1 mm. The formulation of the material for capsule administration could also be as a powder, lightly compressed plugs or even as tablets. The capsules could be prepared by compression.

The preferred embodiment inhibitors DMAT and TBB can be used and administered in a variety of forms, vehicles, and concentrations. Generally, the preferred embodiment inhibitors are used in conjunction with a vehicle such as DMSO, however a wide array of other vehicles may be employed. The inhibitor DMAT can be used so as to achieve in vivo or ex vivo concentrations in the vicinity of the cells of interest, ranging from as low as 0.1 μM to as high as 1,000 μM or more, however a preferred concentration range is from about 1 μM to about 100 μM and more preferably, from about 10 μM to about 50 μM. Similarly, the inhibitor TBB can be used so as to achieve in vivo or ex vivo concentrations in the vicinity of the cells of interest, ranging from as low as 0.1 μM to as high as 1,000 μM or more, however a preferred concentration range is from about 1 μM to about 150 μM and more preferably, from about 15 μM to about 75 μM. Generally, these concentrations are designated as effective amounts.

The instant pharmaceutical composition will generally contain a per dosage unit (e.g., tablet, capsule, powder, injection, teaspoonful and the like) from about 0.001 to about 100 mg/kg. In one embodiment, the instant pharmaceutical composition contains a per dosage unit of from about 0.01 to about 50 mg/kg of compound, and preferably from about 0.05 to about 20 mg/kg. Methods are known in the art for determining therapeutically effective doses for the instant pharmaceutical composition. The therapeutically effective amount for administering the pharmaceutical composition to a human, for example, can be determined mathematically from the results of animal studies.

The present invention provides methods for treating a wide array of diseases, and preferably various types of cancers. Most preferably, the present invention methods can be utilized to treat diseases characterized by over-proliferation of malignant cells, and most notably, chronic myelogenous leukemia, breast cancer, colon cancer, and skin cancer. Indications for treating other types of cancers are described later herein. In a preferred treatment method, an effective amount of one or more preferred CK2α inhibitor(s) is administered to a subject in need of treatment for a duration sufficient to induce proliferation arrest while maintaining a steady cell number. Preferably, the duration ranges from about 1 to about 14 days, and more preferably from about 3 to about 7 days. The one or more preferred inhibitor(s) can be administered multiple times per day so as to produce a preferred effective amount. In addition, it is preferred that prolonged treatment strategies can be defined in accordance with the present invention.

The present invention provides methods for treating a wide array of myeloproliferative disorders, and in particular, for treating chronic myelogenous leukemia. The present invention also provides methods for treating various hematological malignancies, and in particular, for inhibiting hematological malignancies, inducing maturation of malignant megakaryoblasts, inducing thrornbocytosis, reducing platelet production otherwise occurring from malignant megakaryoblasts, and methods for inducing thrombocytopoiesis. And, the present invention provides strategies for treating cancers such as breast cancer, colon cancer, and skin cancer. The present invention also provides methods for treating renal cell carcinoma, bladder cancer, and glioblastoma. In a preferred treatment method, an effective amount of one or more preferred CK2α inhibitor(s) is administered to a subject for a duration sufficient to induce thrombocytopoiesis. Preferably, the duration ranges from about 1 to about 14 days, and more preferably from about 3 to about 7 days. The one or more preferred inhibitor(s) can be administered multiple times per day so as to produce a preferred effective amount. In addition, it is preferred that prolonged treatment strategies can be defined in accordance with the present invention.

The inhibitor compositions may be administered by an initial bolus followed by a continuous infusion to maintain therapeutic circulating levels of drug product. Those of ordinary skill in the art will readily optimize effective dosages and administration regimens as determined by good medical practice and the clinical condition of the individual to be treated. The frequency of dosing will depend on the pharmacokinetic parameters of the agents and the route of administration. The optimal pharmaceutical formulation will be determined by one skilled in the art depending upon the route of administration and desired dosage, see, for example, Remington's Pharmaceutical Sciences, pp. 1435-1712. Such formulations may influence the physical state, stability, rate of in vivo release, and rate of in vivo clearance of the administered agents. Depending on the route of administration, a suitable dose may be calculated according to body weight, body surface area or organ size. Further refinement of the calculations necessary to determine the appropriate dosage for treatment involving each of the above mentioned formulations is routinely made by those of ordinary skill in the art without undue experimentation, especially in light of the dosage information and assays disclosed herein, as well as the pharmacokinetic data observed in human clinical trials. Appropriate dosages may be ascertained by using established assays for determining blood level dosages in conjunction with an appropriate physician considering various factors which modify the action of drugs, e.g., the drug's specific activity, the severity of the indication, and the responsiveness of the individual, the age, condition, body weight, sex and diet of the individual, the time of administration and other clinical factors. As studies are conducted, further information will emerge regarding the appropriate dosage levels and duration of treatment for various indications involving aberrant proliferation of hematopoietic cells.

As used herein, the term “effective amount” means a dosage sufficient to produce a desired or stated effect.

As used herein, the term “leukemia” generally refers to cancers that are characterized by an uncontrolled increase in the number of at least one leukocyte and/or leukocyte precursor in the blood and/or bone marrow. Leukemias including but not limited to acute lymphoblastic leukemia (ALL); acute myeloid leukemia (AML); chronic lymphocytic leukemia (CLL); chronic myelogenous leukemia (CML); and, hairy cell leukemia are contemplated. “Leukemic cells” typically comprise cells of the aforementioned leukemias.

The methods of the invention may be applied to cell populations in vivo or ex vivo. “In vivo” means within a living individual, as within an animal or human. In this context, the methods of the invention may be used therapeutically in an individual, as described herein.

“Ex vivo” means outside of a living individual. Examples of ex vivo cell populations include in vitro cell cultures and biological samples including but not limited to fluid or tissue samples obtained from individuals. Such samples may be obtained by methods well known in the art. Exemplary biological fluid samples include blood, cerebrospinal fluid, urine, saliva. Exemplary tissue samples include tumors and biopsies thereof. In this context, the invention may be used for a variety of purposes, including therapeutic and experimental purposes. Information gleaned from such use may be used for experimental purposes or in the clinic to set protocols for in vivo treatment. Other ex vivo uses for which the invention may be suited are described below or will become apparent to those skilled in the art. Ex vivo applications include in vitro applications, studies, and investigations.

It will be appreciated that the treatment methods of the invention are useful in the fields of human medicine and veterinary medicine. Thus, the individual to be treated may be a mammal, preferably human, or other animals. For veterinary purposes, individuals include but are not limited to farm animals including cows, sheep, pigs, horses, and goats; companion animals such as dogs and cats; exotic and/or zoo animals; laboratory animals including mice, rats, rabbits, guinea pigs, and hamsters; and poultry such as chickens, turkeys, ducks, and geese.

“Pharmaceutically acceptable salts” means any salts that are physiologically acceptable insofar as they are compatible with other ingredients of the formulation and not deleterious to the recipient thereof. Some specific preferred examples are: acetate, trifluoroacetate, hydrochloride, hydrobromide, sulfate, citrate, tartrate, glycolate, oxalate.

Administration of prodrugs is also contemplated. The term “prodrug” as used herein refers to compounds that are rapidly transformed in vivo to a more pharmacologically active compound. Prodrug design is discussed generally in Hardma et al. (Eds.), Goodman and Gilman's The Pharmacological Basis of Therapeutics, 9th ed., pp. 11-16 (1996). A thorough discussion is provided in Higuchi et al., Prodrugs as Novel Delivery Systems, Vol. 14, ASCD Symposium Series, and in Roche (ed.), Bioreversible Carriers in Drug Design, American Pharmaceutical Association and Pergamon Press (1987).

The inhibitors of the invention may be covalently or noncovalently associated with a carrier molecule including but not limited to a linear polymer (e.g., polyethylene glycol, polylysine, dextran, etc.), a branched-chain polymer (see U.S. Pat. Nos. 4,289,872 and 5,229,490; PCT Publication No. WO 93/21259), a lipid, a cholesterol group (such as a steroid), or a carbohydrate or oligosaccharide. Specific examples of carriers for use in the pharmaceutical compositions of the invention include carbohydrate-based polymers such as trehalose, mannitol, xylitol, sucrose, lactose, sorbitol, dextrans such as cyclodextran, cellulose, and cellulose derivatives. Also, the use of liposomes, microcapsules or microspheres, inclusion complexes, or other types of carriers is contemplated.

Other carriers include one or more water soluble polymer attachments such as polyoxyethylene glycol, or polypropylene glycol as described U.S. Pat. Nos. 4,640,835, 4,496,689, 4,301,144, 4,670,417, 4,791,192 and 4,179,337. Still other useful carrier polymers known in the art include monomethoxy-polyethylene glycol, poly-(N-vinyl pyrrolidone)-polyethylene glycol, propylene glycol homopolymers, a polypropylene oxide/ethylene oxide co-polymer, polyoxyethylated polyols (e.g., glycerol) and polyvinyl alcohol, as well as mixtures of these polymers.

The present invention further provides kits for disease diagnosis, prognosis, risk assessment, and/or treatment efficacy determination. Such kits are useful in a clinical setting for use in diagnosing a patient for a disease, monitoring the disease progression, testing patient's samples (e.g. biopsied), for example, to determine or predict if the patient's disease (e.g., cancer) will be resistant or sensitive to a given treatment or therapy with a drug, compound, chemotherapy agent, or biological treatment agent.

### Testing

The hypotheses presented herein were evaluated by a two pronged approach: 1) studies using direct inhibition of malignant cells growth in culture and xenografts in mice; and 2) studies to identify the molecular mechanism by which CK2 inhibition induces arrest of proliferation and apoptosis only in malignant cells. Cell biology experiments aimed to identify nuclear proteins responsible for cell growth were employed. Four cell lines presented herein were used plus several other cell lines (renal, bladder, and brain).

### Testing Procedures

The present inventor was interested in determining the effect of CK2α inhibition on malignant megakaryoblasts, with specific inhibitors. For this, the MEG-01 cell line was selected and characterized as being early stage megakaryoblasts with Philadelphia positive chromosome, isolated from a patient with CML, in blast crisis. These cells are extremely malignant with an increased proliferation rate.

Cell Culture. MEG-01 megakaryoblastic cell line, was a generous gift from Dr. P. B. Tracy, (Department of Biochemistry, University of Vermont, College of Medicine, Burlington, Vt., USA). MEG-01 cells were also purchased from American Tissue Culture Collection (Manassas, Va.). Cells were maintained in an incubator, with a humidified atmosphere of CO₂ 5%, and at 37° C. Cell culture media was RPMI 1640 1× with L-Glutamine (2 mM) from Central Cell Services, Media Lab, (Lerner Research Institute, Cleveland Clinic, Cleveland, USA), and adjusted to contain 10 mM HEPES (Invitrogen, Carlsbad, Calif., USA), 1.0 mM Sodium Pyruvate (Invitrogen, Carlsbad, Calif., USA), 10% heat-inactivated fetal bovine serum (Invitrogen, Carlsbad, Calif., USA), and Penicillin/Streptomycin (Invitrogen, Carlsbad, Calif., USA). Cells were seeded at 2×10⁵ cells/ml, media was renewed and cell number adjusted two times per week.

Cell Treatments. Cells were treated with the preferred embodiment CK2α inhibitors, 4,5,6,7-Tetrabromobenzotriazole (TBB), and 2-Dimethylamino-4,5,6,7-tetrabromo-1H-benzimidazole (DMAT). The vehicle for DMAT and TBB was dimethylsulfoxide (DMSO). As a differentiation control, besides the untreated control (DMSO only), Phorbol 12-myristate 13-acetate (PMA) was used. TBB, DMAT, PMA and DMSO were purchased from Calbiochem (EMD Biosciences San Diego, Calif., USA). Apoptosis and viability assays were performed to choose the non-cytotoxic concentrations of TBB and DMAT that have a significant effect. At the beginning of each treatment cells were counted with a hemacytometer and were split to approximately 2×10⁵ cells/ml. To assess the effect of CK2 inhibitors, the treatment lengths went up to 4 days without splitting.

Proteins, peptides used in platelets function assays and apoptosis assay. Human fibrinogen and the peptides RGD, RGDS and TRAP, were from Sigma (St Louis, Mo., USA). CD62P (anti human P-Selectin monoclonal antibody) conjugated with FITC, CD41a (anti human α_{IIb}β₃ monoclonal antibody) conjugated with RPE, PAC-1 antibody conjugated and Annexin V conjugated with FITC and PI (Annexin V-FITC and PI Apoptosis Kit I), were all purchased from BD Biosciences, Bedford, Mass., USA. Human thrombin was purchased from Haematologic Technologies Inc, Essex Junction, Vt., USA.

Flow cytometric analysis. Cells were analyzed using a FACSCalibur flow cytofluorometer (Becton-Dickinson), with CellQuestPro ver. 3.3 software. The data was further analyzed with FlowJo ver. 6.2, and WinMDI 2.8 software. Proper gating was performed to characterize each cell population (MEG-01 cells and platelets). The population that corresponds to platelets (small, granulated particles), is PI negative because thrombocytes are anucleated cells (only the viable cells were considered) and were distinguished by their capacity to become activated, undergoing shape change in response to agonist and to show phosphatidylserine (PS) exposure when activated. Platelets were further separated from the megakaryocytic cells, by differential centrifugation, considering the size difference between these cell populations (1-5 μm for platelets and 35-150 μm for megakaryocytic cell line MEG-01) and analyzed separately. Voltage and channels settings were adjusted accordingly. Analyzed values were obtained with WinMDI ver. 2.8.

Apoptosis Assays using Flow Cytometry with Annexin V-FITC and propidium iodide (PI). For induction of typical apoptosis, cells were grown in the presence of 1 μM staurosporine (Sigma-Aldrich) for 6 hours. The staurosporine treated cells were stained as follows: control 1 with PI, control 2 with Annexin V-FITC and control 3 both PI and Annexin V-FITC in order to have the brightest controls for compensation and proper collection of the flow cytometry data. Cell necrosis was induced by heat shock (65° C. for 30 minutes). The stained control, both PI and Annexin V-FITC labeled, was then used as the reference in establishing the level of apoptosis induced by the treatments. Data was collected on logarithmic modes, two-colors. Annexin V-FITC corresponds to FL1 H channel, and PI to FL3H or FL2H channels.

Annexin V-FITC and PI apoptosis assay staining protocol. Apoptosis Annexin V-FITC and PI kit from BD Biosciences, CA, USA, was used. 10⁵-10⁶ cells were stained with 50 μg/ml PI and with 0.5 μg/ml FITC-labeled Annexin V using the staining protocol provided by the supplier. Samples were then analyzed immediately by flow cytometry.

Flow cytometric DNA content assessment assay using PI/RNAse A. Cells were serum starved in order to synchronize them in the GO phase. Treatment with DMAT 10 μM for a period of 4 days was performed and then cells were collected for further processing. Cells were fixed in 80% cold Ethanol/PBS added drop-wise. Before PI staining cells were washed with sterile, RNAse, DNAse free, PBS buffer. Fixed cells were incubated with 50 μg/ml PI and 100 μg/ml RNAse A-I in hypotonic citrate staining buffer for 30 minutes in dark, at room temperature.

RPE-CD41a immunophenotyping of MEG-01 cells. CD41a is the antigen for α_{IIb}β₃ complex and it is found on platelets and platelet precursors, including MEG-01 cell line. α_{IIb}β₃ complex is a marker of differentiation for megakaryocytes. The staining protocol provided by BD Biosciences was used. Briefly, cells were washed and resuspended in 1×PBS with 0.1% FBS, 0.01% NaN₃, and 0.22 μm filtered buffer. Cells were counted and adjusted to 10⁶ cells/ml and 20 μl of RPE-CD41a was used for 180 μl cell suspension. RPE-CD41a stained cells were collected on FL2H channel and gating was performed on FSC and SSC logarithmic modes. The unstained control signal was subtracted from the stained cell signal, in order to measure the staining of the cells without background noise.

Platelets isolation from culture. Platelets were separated from the megakaryocytic cells, by differential centrifugation and analyzed separately. Suspension cells were centrifuged first at 100 g-150 g for 15 minutes, and then the platelets rich supernatant was kept and centrifuged again at 800 g for 15 minutes. For impeding artefactual aggregation of platelets in the control, EDTA and RGD or RGDS were added in the cell suspension from the beginning of the centrifugations, and with each centrifugation step. 1 mg/ml RGD or RGDS and 10 mM EDTA final concentrations, were used for negative control (inactivated) or resting platelets. Platelets were activated with different agonists: 100 nM PMA, 1 μg/ml TRAP and 0.5 U/ml human a-thrombin. For most of assays TRAP 1 μg/ml was used. For scanning electron microscopy experiment (formation of the fibrin clot) human a-thrombin was used. For both sample sets, activated (with TRAP) and inactivated (EDTA, RGD), platelets collected from MEG-01 cultures incubated with 10 μM DMAT for 4 days were utilized.

PAC-I-FITC binding due to platelet activation flow cytometric assay. Monoclonal antibody PAC-1 recognizes an epitope on the glycoprotein α_{IIb}β₃ of activated platelets. PAC-1 binds only to the activated platelets. PAC-1 will not bind EDTA and RGD or RGDS treated platelets. 20 μl PAC-1-FITC were used for 5 μl fresh platelets suspension, in Tyrode's buffer with CaCl₂, Activation of platelets with 1 μg/ml TRAP was performed for 10 minutes. The protocol provided by BD Biosciences was used for staining.

CD62P-FITC exposure due to platelet activation flow cytometric assay. CD62P is a monoclonal antibody that recognizes an epitope on P-Selectin. P-Selectin is exposed as response to agonist and is a specific sign of platelet activation. 20 μl CD62P-FITC were used for 5 μl fresh platelets suspension, in Tyrode's buffer with CaCl₂. Activation of platelets with 1 μg/ml TRAP was performed for 10 minutes. Incubation was performed in the dark at room temperature for 30 minutes, as recommended by BD Biosciences.

Fibrinogen-Alexa Fluor 488 binding to platelets flow cytometric assay. Human fibrinogen was conjugated with Alexa Fluor 488 fluorochrome (F-13191, from Molecular Probes, OR, USA) following recommended Molecular Probes procedure. Final concentration of fibrinogen conjugated to Alexa Fluor 488 was determined spectrophotometrically. Activation of platelets with 1 μg/ml TRAP was performed for 10 minutes. 300 nM (as a final concentration) labeled fibrinogen was incubated with platelets suspension for 30 minutes, in dark, at room temperature.

Annexin V-FITC for phosphatidylserine (PS) exposure on platelets. For this assay, Annexin V-FITC (BD Biosciences) was used, as recommended by the manufacturer. Following activation for 10 minutes with agonist (1 μg/ml TRAP), incubation of the platelets was performed in the dark, at room temperature for 30 minutes.

Viability (proliferation) assay, Trypan blue exclusion. Cells were counted using a Neubauer hemacytometer. Trypan blue dye was used according to the manufacturer (Sigma-Aldrich). DMSO, which is the vehicle for TBB, DMAT and PMA, was used as a mock control, considering the highest amount that was used as a vehicle for TBB and DMAT.

Anchorage independence in “soft agar” assay. Agarose (Promega) was mixed with MEG-01 growth media RPMI1640 1× with 10% FBS. Cells were grown in 12 wells dishes at 37° C., 5% CO₂, 90% humidity in a VWR incubator. Colonies formation was observed and micrograph images were taken using an Olympus CK40 microscope. Cells were observed after one week. The control (DMSO) samples were compared with the samples grown in the presence of 25 μM DMAT. Media was renewed on the top of the agar every 4 days and DMAT treatment was performed every time. 85 colonies from 30 images were analyzed. Measurements of the colonies areas were performed using the NIH Image software ver. 1.63 for MacOS 9.

Light microscopy (phase contrast) and DAPI fluorescence microscopy. High quality pictures and live imaging were obtained at Cleveland Clinic Imaging Core (Cleveland, Ohio). DAPI staining of cultured MEG-01 cells was performed with fresh cells. For live imaging (observing a single cell for a 24 hour period of time) of the thrombocytopoiesis process, MEG-01 suspension cells were made adherent by using Fibronectin (FN) coated culture dishes. FN was used, at 5 μg/cm² and incubated at 37° C. for 1 h, as recommended by the supplier (BD Biosciences, Bedford, Mass., USA).

Scanning electron microscopy (SEM). Cell preparation and pictures were performed at the Microscopy Core Facility (Cleveland Clinic). Cells were grown for 4 days in the presence of 10 μM DMAT and then collected by differential centrifugation. Cells were fixed in glutaraldehyde and then further processed at the Core. The clot was prepared with platelets collected from MEG-01 cultures treated with 10 μM DMAT. Human thrombin 0.5 U/ml for 15 minutes was used to activate them.

Statistical Analysis. Error bars are standard deviations (SD). Experiments were performed at least in triplicates. Statistical analysis and graphing were performed using GraphPad, Prism software ver. 2.01. One-Way ANOVA Test-Repeated Measures followed by Dunnett's Multiple Comparison Test (which compares all treatment columns vs. the control column) or student t-test were also performed. p<0.05 (*) was considered significant, p<0.01 (**) very significant and p<0.001 (***) extremely significant.

### Cell Lines MEG-01

Creation of MEG-01 xenografts. Immunodeficient male athymic nude nu/nu mice were used, provided and housed by Dr. Lindner, D J from Taussig Cancer Center, Cleveland Clinic and Case Western Reserve University. The mice were checked every day. The mice were housed in filtered air flow cabinets with autoclaved bedding at a density of 5 mice/cage. They were fed autoclaved Purina Lab Rodent Chow® 5010 and HCl-acidified distilled water ad libitum and were placed in rooms with controlled temperature, humidity and 12-hr light-dark cycles. Procedures involving animals and their care were conducted in conformity with the institutional guidelines that are in compliance with national and international laws and policies (EEC Council Directive 86/609, OJL 358, Dec. 1, 1987, and the National Institutes of Health Guide for the Care and Use of Laboratory, Animals, NIH Publication 85-23, 1985).

For engraftment, cells in cell culture media were injected. MEG-01 myeloid blast crisis cells (10×10⁶ cells/100 μl in cell culture media (RPMI 1640 1×, 10% FBS) were injected subcutaneously into the lower flanks of mice (left and right). Cells were counted with a hemacytometer using Trypan blue (only live cells were counted).

After 10 days the mice developed tumors large enough to start the treatment. Tumors were visible after 6-7 days from inoculation. The average weight of the mice typically ranged 30-35 g. Treatment with DMAT was started when tumors were at least 100-200 mm³ volumes (prolate spheroid). Tumor volumes were calculated as prolate spheroid (4/3*π*(a)²*(b), where “2a” is the minor axis and “2b” is the major axis of the prolate spheroid. “2a” and “2b” were measured with a caliper (mm). Animals were treated with DMAT for approximately 2 weeks. When tumor volumes reached a size unacceptable with the IACUC protocols, animals were sacrificed in a CO₂ euthanasia chamber. Tumors were collected for further histological analysis.

To assess the statistical significance of difference between pairs of means of tumor volumes, student's two-tailed t test was used. p<0.05 was considered significant (*).

In vivo therapy with DMAT of MEG-01 xenografts. Therapy with DMAT was done in two trials. Tumor diameters were measured using a caliper and tumor volume was calculated using the prolate-spheroid formula. DMAT in DMSO as well as just DMSO as a control were administered by injection subcutaneous in the neck (exogenous from the tumor). Tumor measurements indicate if DMAT induces tumor ablation in MEG-01 xenograft.

In vivo toxicity study for DMAT. A male nude mouse (without tumors) was treated with DMAT to determine a toxicity level. A saturated solution of DMAT in DMSO was inoculated daily in the neck subcutaneously (100 μl of 100 mg/ml DMAT in DMSO). Therefore this animal received 10 mg DMAT per day. After 2 weeks this animal was sacrificed and organs were collected for further analysis.

Treatment of MEG-01 murine xenografts with DMAT. First trial with n=4 (2 tumors on left and right flanks) was started when tumor volumes were quite large (500-650 mm³). DMAT in DMSO was administered daily as 2 mg/animal, subcutaneously into the neck (in the neck fat pad), exogenous from the tumors. Injection was done as 50 μl DMAT 40 mg/ml per animal per day. The trial length was for 2 weeks. Tail-bleeding and blood collection were performed in this animal before sacrifice.

Second trial with n=6 (2 tumors on left and right flanks) was started when tumor volumes were 100-200 mm³. DMAT in DMSO was administered daily as 3 mg/animal, subcutaneously into the neck (in the neck fat pad), exogenous from the tumors. Injection was done as 50 μl DMAT 60 mg/ml per animal per day. The trial length was for 2 weeks.

At the end of the trial, tail-bleeding times assay was performed and blood was collected for blood counts from live and anesthetized mice. After these experiments were completed mice were sacrificed and tumors and organs were collected. Samples were fixed in formalin for further processing.

A batch of Control mice, with MEG-01 tumors, n=4 and respectively n=6 (treated with 50 μl DMSO, which is the vehicle for DMAT) was compared (as tumor volumes and tissue, tail-bleeding and blood counts) in parallel with the DMAT treated mice.

Hematoxylin & eosin staining of tumor tissue and organs. After euthanasia with CO₂, mice were supposed to necropsy. Tumors were collected from under the skin from both flanks and were measured for the last time and then fixed in formalin fixative. Spleen, liver, kidney, brain, lungs and legs were collected and fixed. Spleens were also measured (as length, m). Fresh tissue was immersed immediately into liquid nitrogen and kept frozen at −80° C. Fixed tissue was embedded in paraffin and next processed for H&E (the basic dye hematoxylin, and the alcohol-based synthetic material, eosin) by the Cleveland Clinic Histology Core facility. Sections (4-μm thick) were stained with hematoxylin and eosin and evaluated for pathologic changes in a blinded fashion. H&E staining gives morphological information (vascularization, normal proliferating tissue, necrosis and apoptosis of the tissue).

Mice blood collection and blood counts. Blood counts are dependent upon the method and time of blood collection. Whole blood was collected from the retro-orbital sinus (under the eye) of anesthesiated mice (both DMSO treated and DMAT treated batch). EDTA and prostaglandin E1 (PGE1) were used at collection to prevent clotting during blood collection. 500 μl mice whole blood with 100 μl anticoagulant were used for counting (a 1:5 dilution). A hematological analyzer was used for this. Samples were compared (gated) with normal mice (C57BL strain).

Tail-bleeding assay. Tail-bleeding times are important to investigate whether the platelets could establish hemostasis in vivo. Platelet aggregation and clot retraction in response to physiologic agonists adenosine diphosphate (ADP), epinephrine, and thrombin will affect tail-bleeding times.

Normal tail-bleeding times are an average of 1.5-2 minutes in C57BL mouse strain.

Pre-warm tubes of saline (PBS) at 37° C. and maintained at this temperature during the measurements were used. Inhalation of isoflurane vapor or, alternatively, intraperitoneal injection of avertin was used to induce general anesthesia. Using a sharp new razor or scalpel blade, tails were cut exactly 0.5 cm of the distal tip of the tail of the adult mouse and immediately inserted into the pre-warmed tube of saline. A stopclock was started at this time. The tail was hold gently, near its base, to avoid a “tourniquet effect.” Venous blood flowing into the tube can be observed and can it can be detected when this bleeding stops. The stopclock provides an accurate bleeding time.

### Cell Lines MCF-7, SW-480, and WM-164

Creation of the xenografts MCF-7, SW-480 and WM-164. Immunodeficient male and female athymic nude nu/nu mice were used, provided and housed by Dr. Lindner, DJ from Taussig Cancer Center, Cleveland Clinic and Case Western Reserve University. Mice were checked every day. Mice were housed in filtered air flow cabinets with autoclaved bedding at a density of 5 mice/cage. They were fed autoclaved Purina Lab Rodent Chow® 5010 and HCl-acidified distilled water ad libitum and were placed in rooms with controlled temperature, humidity and 12-hr light-dark cycles. Procedures involving animals and their care were conducted in conformity with the institutional guidelines that are in compliance with national and international laws and policies (EEC Council Directive 86/609, OJL 358, Dec. 1, 1987, and the National Institutes of Health Guide for the Care and Use of Laboratory, Animals, NIH Publication 85-23, 1985).

For engraftment, cells in cell culture media were injected. Cells (4×10⁶ cells/100 μl MCF-7 cells, 2×10⁶ cells/100 μl SW-480 and 3×10⁶ cells/100 μl WM-164) in cell culture media (DMEM F12, 10% FBS) were injected subcutaneously into the lower flanks of mice (left and right). Cells were counted with a hemacytometer using Trypan blue (only live cells were counted).

Tumors were visible after 6-7 days from inoculation. The average weight of the mice used in these sets of experiments was 30-35 g for male mice and 20-25 g for female mice. Female mice must be used for MCF-7 xenografts, because require hormone supplementation (estradiol). This hormone was provided in drinking water with glucose to be more palatable.

Treatment with DMAT was started when tumors were at least 100-200 mm³ volumes (prolate spheroid). Tumor volumes were calculated as prolate spheroid (4/3*π*(a)2*(b), where “2a” is the minor axis and “2b” is the major axis of the prolate spheroid. “2a” and “2b” were measured with a caliper (mm). Animals were treated with DMAT for approximately 2 weeks (when tumor volumes reached a size unacceptable with the IACUC protocols, animals were sacrificed in a CO₂ euthanasia chamber). Tumors were collected for further histological analysis. To assess the statistical significance of difference between pairs of means of tumor volumes, student's two-tailed t test was used. p<0.05 was considered significant (*).

In vivo therapy with DMAT of MCF-7 xenografts. Therapy with DMAT was done in two trials (n=4 both, with 2 tumors per mice). Mice were supplemented estradiol in drinking water. Tumor diameters were measured using a caliper and tumor volume was calculated using the prolate-spheroid formula. DMAT in DMSO as well as just DMSO as a control were administered by injection subcutaneous in the neck (exogenous from the tumor). Tumor measurements will show if DMAT induces tumor ablation in MCF-7 xenografts.

In vivo therapy with DMAT of SW-480 xenografts. Therapy with DMAT was done in one trial (n=4 both, with 2 tumors per mice). Tumor diameters were measured using a caliper and tumor volume was calculated using the prolate-spheroid formula. DMAT in DMSO as well as just DMSO as a control were administered by injection subcutaneous in the neck (exogenous from the tumor). Tumor measurements will show if DMAT induces tumor ablation in SW-480 xenografts.

In vivo therapy with DMAT of WM-164 xenografts. Therapy with DMAT was done in one trial (n=4 both, with 2 tumors per mice). Tumor diameters were measured using a caliper and tumor volume was calculated using the prolate-spheroid formula. DMAT in DMSO as well as just DMSO as a control were administered by injection subcutaneous in the neck (exogenous from the tumor). Tumor measurements will show if DMAT induces tumor ablation in WM-164 xenografts.

Hematoxylin & eosin staining of tumor tissue and organs. After euthanasia with CO₂, mice were supposed to necropsy. Tumors were collected from under the skin from both flanks and were measured for the last time and then fixed in formalin fixative. Spleen, liver, kidney, brain, lungs and legs were collected and fixed. Spleens were also measured (as length, m). Fresh tissue was immersed immediately into liquid nitrogen and kept frozen at −80° C. Fixed tissue was embedded in paraffin and next processed for H&E (the basic dye hematoxylin, and the alcohol-based synthetic material, eosin) by the Cleveland Clinic Histology Core facility. Sections (4-μm thick) were stained with hematoxylin and eosin and evaluated for pathologic changes in a blinded fashion. H&E staining gives morphological information (vascularization, normal proliferating tissue, necrosis and apoptosis of the tissue).

### Results of Testing

### Effect of Preferred Inhibitors Upon Chronic Myelogenous Leukemia (MEG-01)

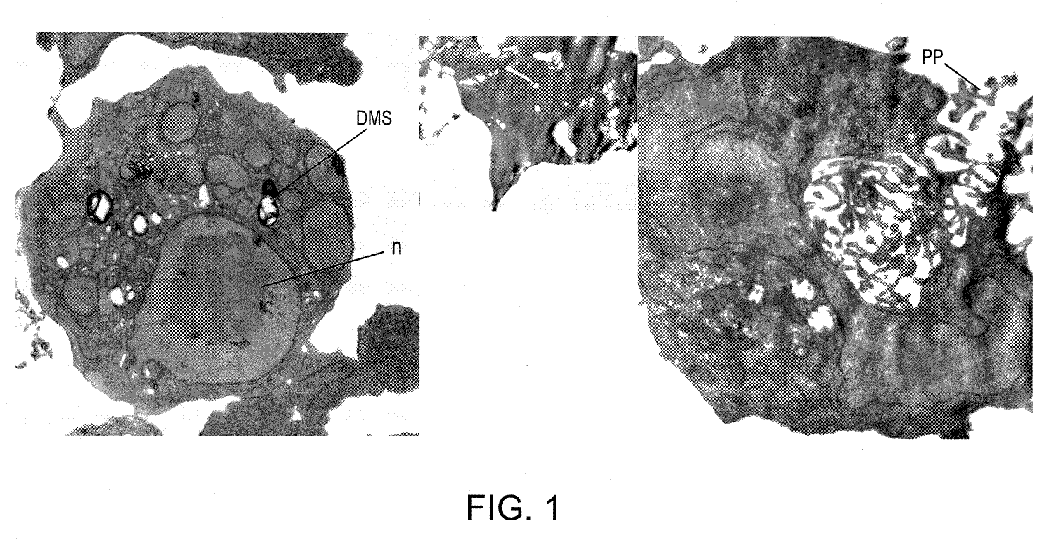

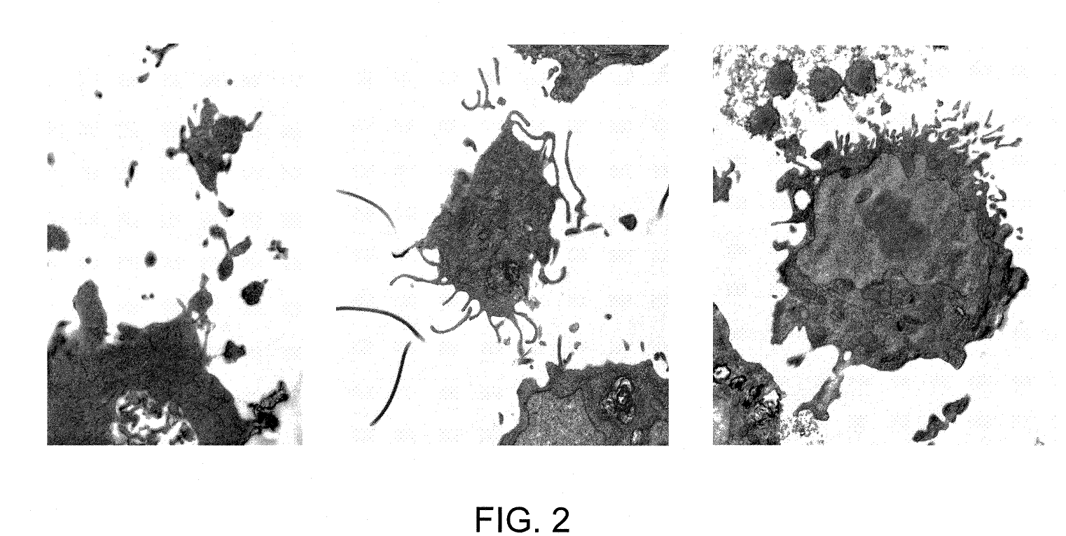

In this study, the inhibition of CK2α was investigated. Specifically, inhibition of CK2α with the preferred inhibitors induced thrombocytopoiesis, forming proplatelets from a demarcation membrane system. Referring to FIG. 1, the photograph on the left illustrates a MEG-01 cell prior to thrombocytopoiesis. The photograph on the right illustrates a MEG-01 cell undergoing thrombocytopoiesis, at 20,000× magnification, and 60 kV. The MEG-01 cells in the photographs of FIG. 1 were treated with DMAT 10 μM for 4 days. FIG. 2 includes photographs of MEG-01 cells undergoing thrombocytopoiesis, at 20,000× magnification, and 60 kV. The cells were treated with DMAT 10 μM for 4 days.

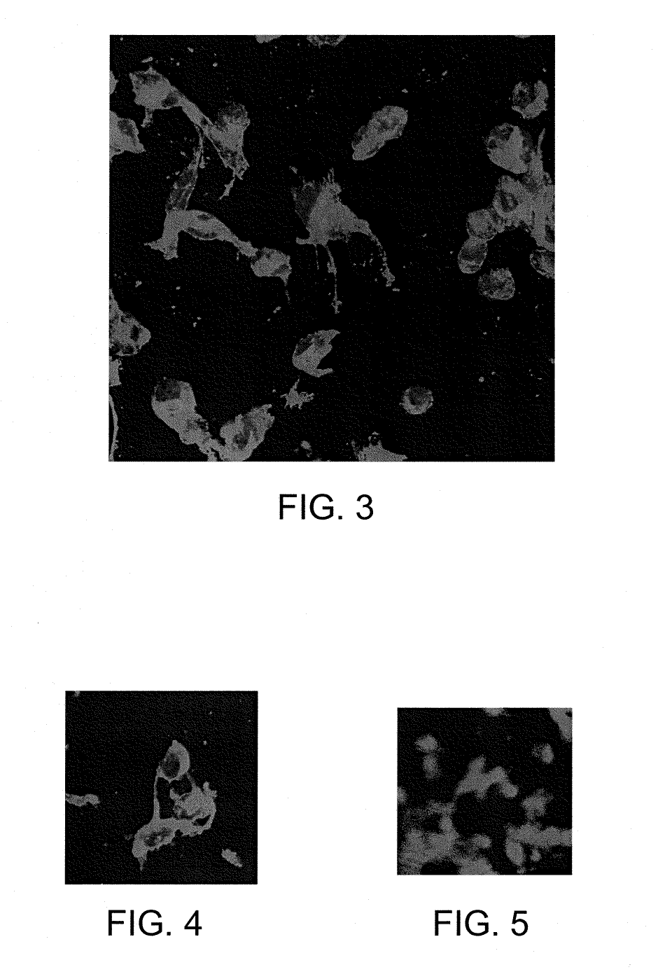

FIGS. 3-5 illustrate MEG-01 cells producing platelets after treatment with a preferred inhibitor. FIG. 3 shows MEG-01 cells producing platelets, the image obtained from confocal microscopy (Phalloidin-Alexa Fluor 488 in DAPI mounting media) at 63× magnification. FIG. 4 shows MEG-01 cells producing platelets, with Phalloidin only, at 63×. FIG. 5 shows MEG-01 cells producing platelets, with Phalloidin only, at 63× and 8× digital zoom.

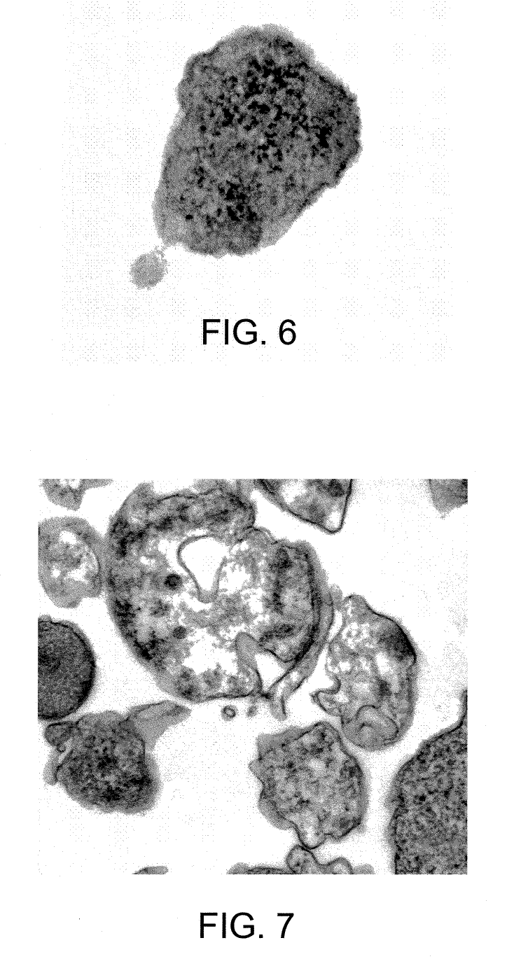

FIGS. 6 and 7 illustrate that platelets produced from the MEG-01 cell line, treated with the preferred embodiment inhibitor DMAT, respond to thrombin. Specifically, FIG. 6 is a photograph of a resting platelet-particle from MEG-01 cells, at 10,000× and 60 kV. FIG. 7 is a photograph of an activated platelet-particle from MEG-01 cells, at 20,000× and 60 kV. These photographs demonstrate that platelets released from a MEG-01 cell line, due to CK2α inhibition, can and do, respond to thrombin. Platelets obtained from MEG-01 cells following treatment with DMAT are functional, in that the platelets form aggregates and release their content in response to human thrombin, at 0.5 U/ml.

Control of malignant potency. The goal of this study was to further investigate the effect of CK2α inhibition on malignant megakaryoblasts. As previously noted, to accomplish this goal a MEG-01 cell line was selected, which is characterized as early stage megakaryoblasts with Philadelphia positive chromosome. The cells were initially isolated from a patient with chronic myelogenous leukemia (CML), in blast crisis. These cells are extremely malignant because of an increased proliferation rate.

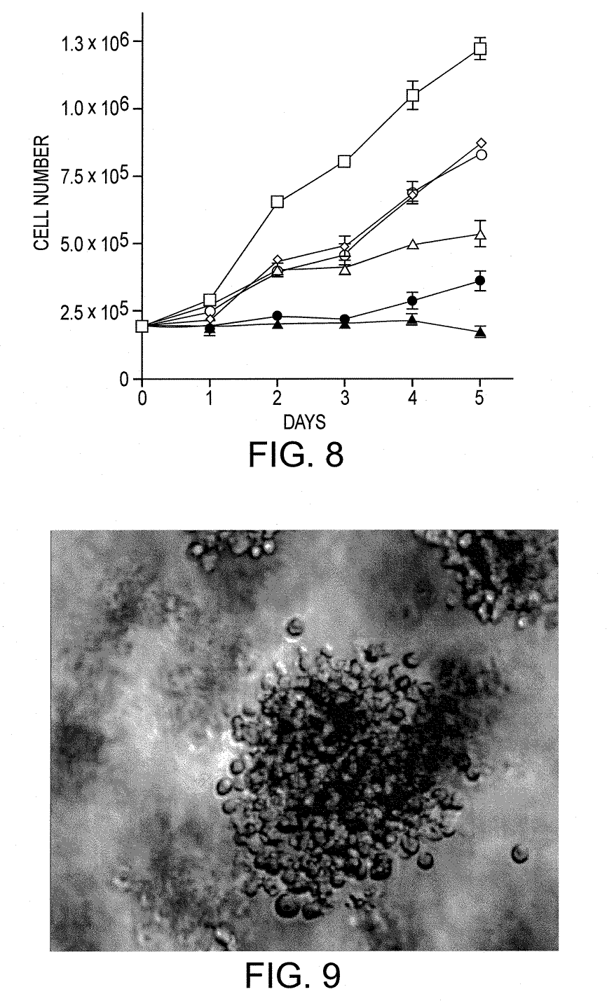

Specifically, the use of CK2 inhibitors DMAT and TBB in MEG-01 cells induces proliferation arrest and decreases the tumorigenicity (anchorage independence) of these malignant megakaryoblasts. FIG. 8 illustrates cell proliferation (viability) assay, with Trypan Blue exclusion of five days of treatment of MEG-01 cells. Open squares represent control untreated, open triangles 50 μM TBB, filled triangles 100 μM TBB, open circles 25 μM DMAT, filled circles 50 μM DMAT and open diamonds PMA 5 nM. Each day quadruplicate measurements were taken and triplicate sets of experiments were considered for the measurements. Specifically, the effect of CK2α inhibitors (TBB and DMAT) was first tested on the proliferation rate of MEG-01 cells using Trypan blue proliferation assay (FIG. 8). Because DMAT and TBB were solubilized in DMSO, initial control experiments were undertaken to establish the effect of DMSO on cell growth. The data demonstrate that DMSO has no effect on MEG-01 cells proliferation rate and apoptosis. In the absence of inhibitor there is a 6-fold increase in cell number (FIG. 8, open squares). In the presence of 5 nM PMA, which is known to induce proliferation arrest and differentiation in MEG-01 cells, a decrease in proliferation was observed (FIG. 8, open diamonds) and the level of decrease was similar to that obtained in the presence of 25 μM DMAT (FIG. 8, open circles) suggesting that DMAT may also induce differentiation. High concentrations of CK2 inhibitors, (50 μM DMAT or 100 μM TBB) maintained a steady number of cells for the 4 days of treatment and induced significant reduction in the proliferation rate of MEG-01 cells (FIG. 8, filled triangles and filled circles, respectively). These data suggest that the inhibitors TBB and DMAT are capable of reversing the proliferating phenotype of MEG-01 cells, without inducing necrosis.

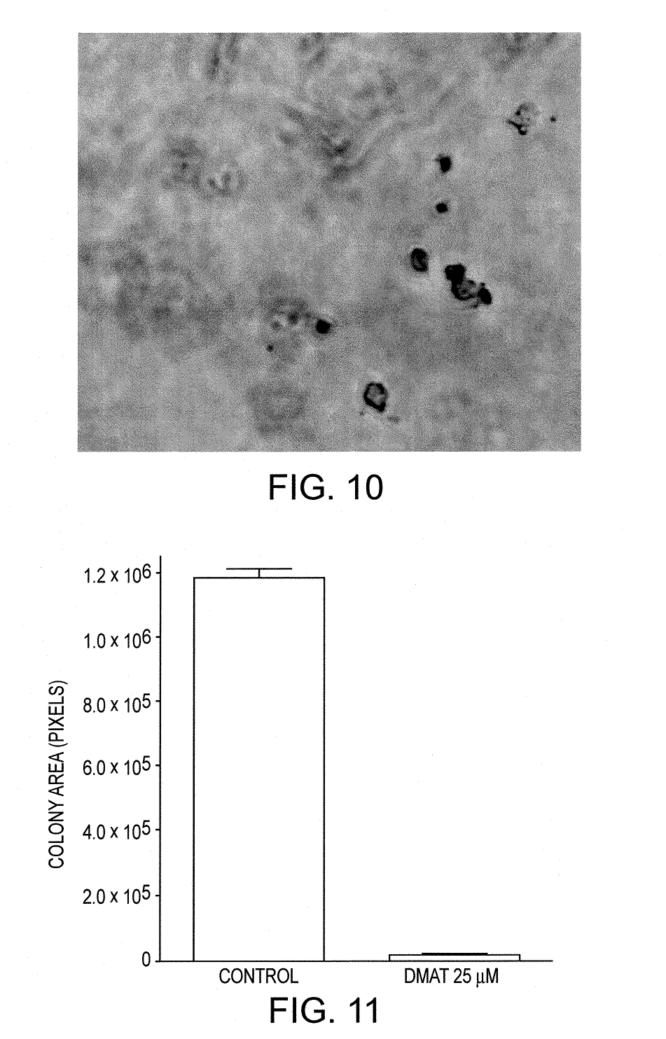

Next, the effect of DMAT on the malignant potential of MEG-01 cells was tested, using anchorage independence assay in soft agar (FIGS. 9-11). FIG. 9 illustrates control untreated colony formation in soft agar by MEG-01 cells, magnification ×20, phase-contrast micrograph. FIG. 10 shows DMAT treated (25 μM) colony formation in soft agar by MEG-01 cells, magnification ×20, phase-contrast micrograph. FIG. 11 illustrates anchorage independence assay in soft agar. Comparison of colonies areas (pixels) between control untreated and DMAT treated MEG-01 cells. Anchorage independence of growth in soft agar assay is strongly connected with tumorigenicity and invasiveness. It has been well established that malignant cells form colonies when grown on soft agar, while non-transformed cells do not grow under similar experimental conditions. The data show that the area and the number of the colonies formed by untreated MEG-01 cells are extensive (FIG. 9), whereas the DMAT-treated MEG-01 cells do not form colonies (FIG. 10). The results in FIG. 11 summarize colony area determined from 30 representative images. These data unequivocally demonstrate that inhibition of CK2α by DMAT eliminates the malignant potential of MEG-01 cells.

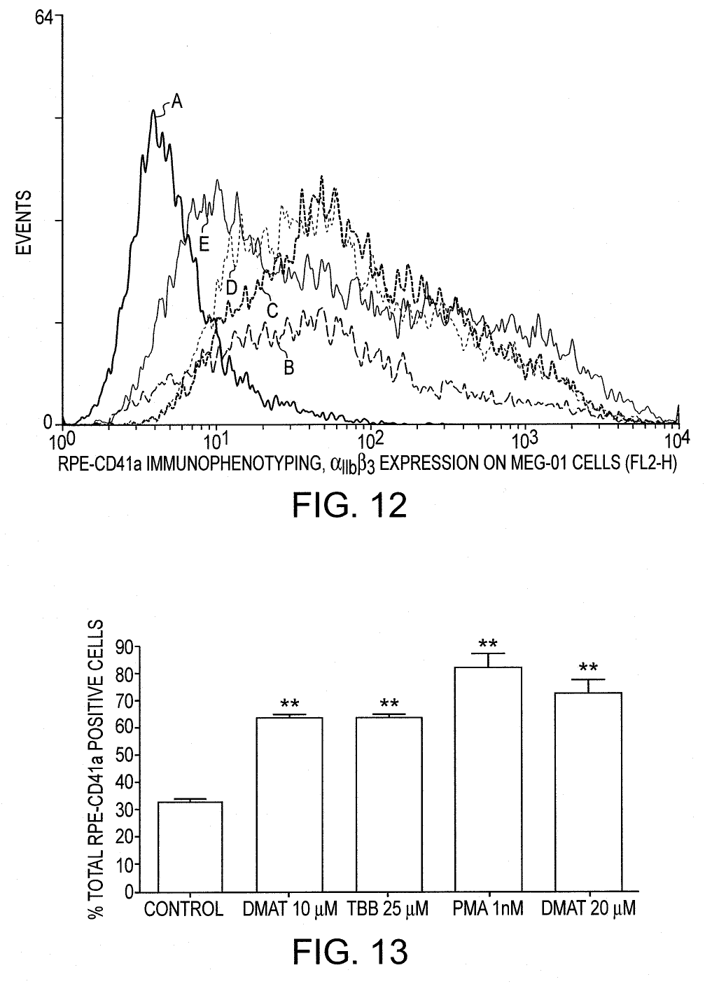

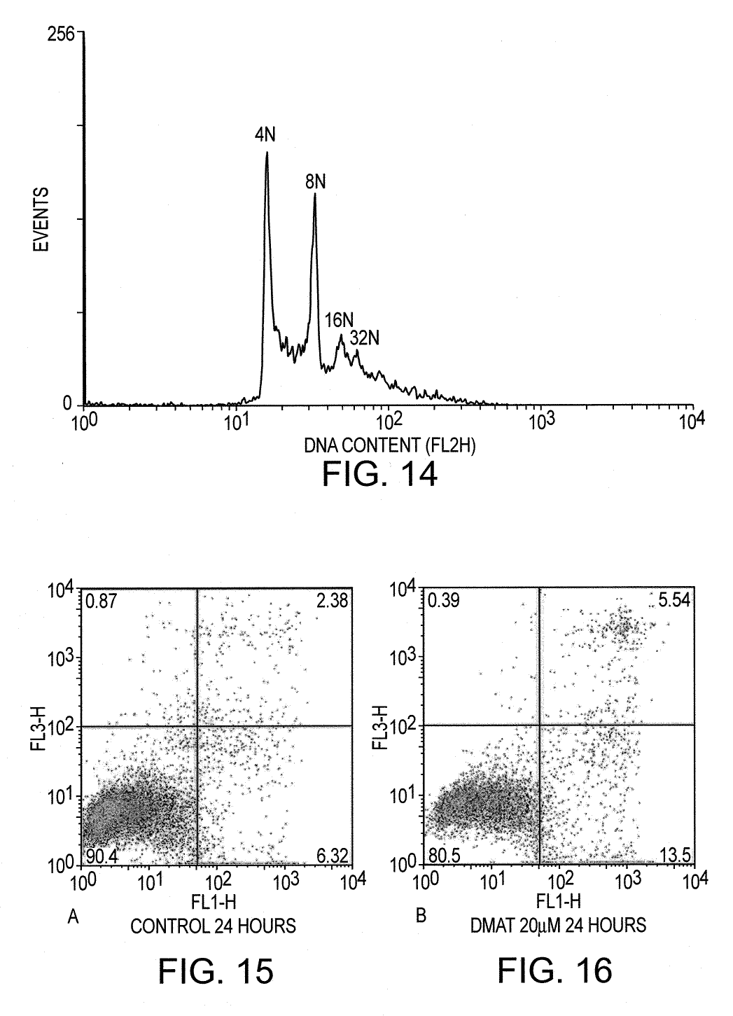

Maturation of MEG-01 megakaryoblasts. Thrombocytopoiesis follows the maturation and differentiation of megakaryoblasts. In order to assess the maturation process of MEG-01 cells in the presence of CK2 inhibitors, flow cytometric immunophenotyping was used, considering the α_{IIb}β₃ integrin as a maturation marker (maturation correlates with increased levels of α_{IIb}β₃). Following incubation with the inhibitor, the levels of expression of α_{IIb}β₃ increase. This increase is correlated with increase in size and differentiation of megakaryoblasts. Maturation (differentiation) of MEG-01 cells due to DMAT is shown in FIGS. 12-14. In FIG. 12, RPECD41a (α_{IIb}β₃ integrin expression) flow cytometric immunophenotyping for DMAT, TBB and PMA treatments versus control untreated is indicated as follows. Histogram shows results from one set of treatments (total relative fluorescence). Control untreated unstained—plot A, control untreated stained—plot B, 10 μM DMAT—plot C, 25 μM TBB—plot D, 1 nM PMA treatments—plot E. FIG. 12 shows that 10 μM DMAT is sufficient to obtain significant maturation levels compared to the control untreated cells. A further increase in the concentration of DMAT (up to 20 μM) induces a slight increase in the maturation level of MEG-01 cells. In FIG. 13, the graph represents total relative fluorescence percentages for RPE-CD41a immunophenotyping, conform analysis of data in WinMDI ver 2.8, from triplicate experiments. FIG. 13 also shows that 20 μM DMAT induced a similar maturation level as 1 nM PMA and 25 μM TBB. In FIG. 14 DNA content analysis is shown of MEG-01 cells treated with 10 μM DMAT for 4 days, as assessed by PI and RNAase A, flow cytometric assay. Finally, DNA content assay demonstrates that MEG-01 cells become polyploid (ploidy higher than 2N) in the presence of 10 μM DMAT. The increase in DNA content and cell size demonstrate that MEG-01 undergo maturation in the presence of CK2α inhibitors treatments. Collectively, the data demonstrate that inhibition of CK2 in MEG-01 cells results in proliferation arrest followed by maturation of the cells.

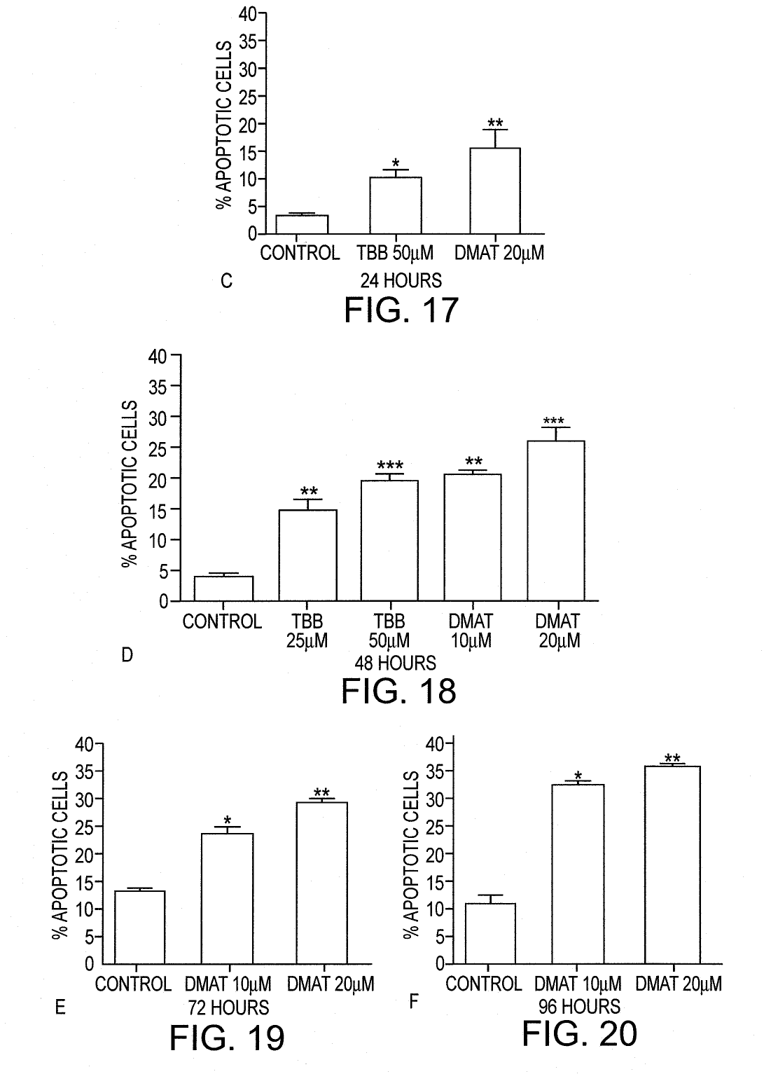

Necrosis versus apoptosis. In order to understand the effect of the inhibitors on MEG-01 cells and verify if the treatment is not cytotoxic, assessment of both apoptosis and necrotic levels, using an assay employing Annexin V was undertaken. Apoptosis and phenotype change induced by CK2 inhibitors (DMAT and TBB) in leukemia megakaryoblasts are dose and time dependent and indicated as follows. Percentages of total apoptotic cells are the sum of (FL1 H+, FL3H−) lower right quadrant, corresponding to early apoptotic cells gate, with (FL1 H+, FL3H+) upper right quadrant, corresponding to late apoptotic cells. Total apoptotic cells percentages and controls were plotted for each treatment set for each day. In FIG. 15, quadrant gating of a control untreated MEG-01 cells after 24 hours (Annexin V-FITC and PI flow cytometric assay). Necrotic cells correspond to the upper left quadrant gate (FL1 H−, FL3H−). In FIG. 16, quadrant gating of cells treated with 20 μM DMAT following 24 hours. FIG. 15 demonstrates that following 24 hours incubation in the absence of CK2 inhibitors, 8.7% of the control untreated cells are apoptotic while 0.87% are necrotic. Following 24 hours treatment with 20 μM DMAT the level of apoptotic cells significantly increased (19%) while the level of necrotic cells remained low 0.39% (FIG. 16). A comparative summary of the results obtained following 24 hours incubation with either TBB or DMAT is provided in FIG. 17. In FIG. 17, the effect of TBB and DMAT treatment on MEG-01 cells apoptosis after 24 hours. In FIG. 18, the effect of TBB and DMAT treatment on MEG-01 cells apoptosis after 48 hours In FIG. 19, the effect of TBB and DMAT treatments on MEG-01 cells apoptosis after 72 hours. In FIG. 20, the effect of TBB and DMAT treatments on MEG-01 cells apoptosis after 96 hours. The results provided in FIGS. 17-20 represent the average found in three independent experiments. The data shown in FIGS. 18-20 demonstrate that the effect is dose dependent and reaches a maximum after four days. Following 96 hours incubation the results obtained with 10 μM DMAT are similar to the results obtained with 20 μM inhibitor. A direct comparison between control cells and DMAT-treated cells establish that treatment with DMAT induces significant apoptosis in MEG-01. These findings also clearly demonstrate that both CK2 inhibitors are not cytotoxic.

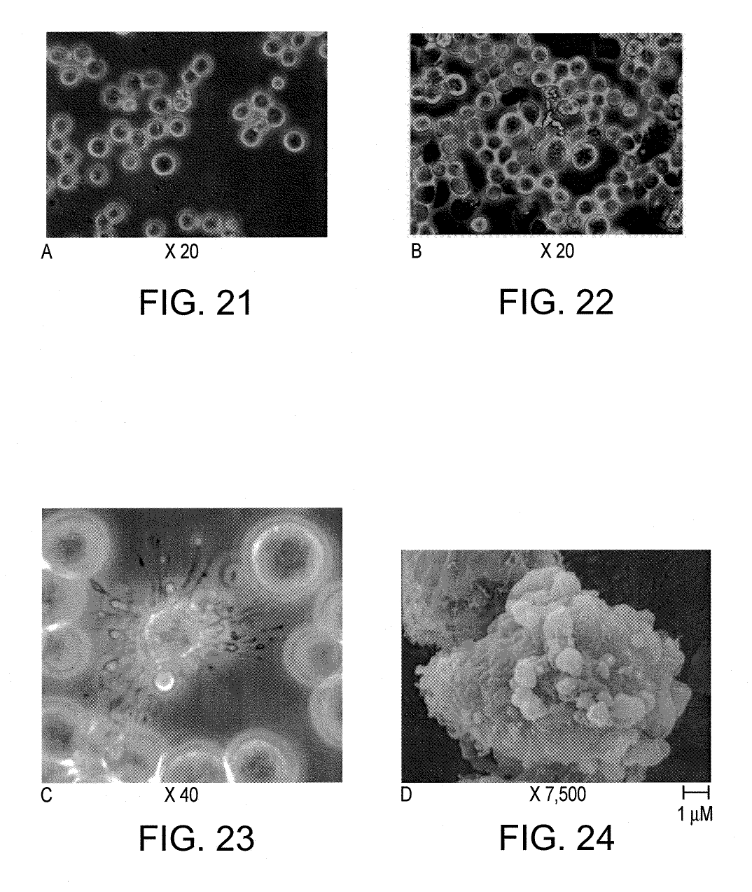

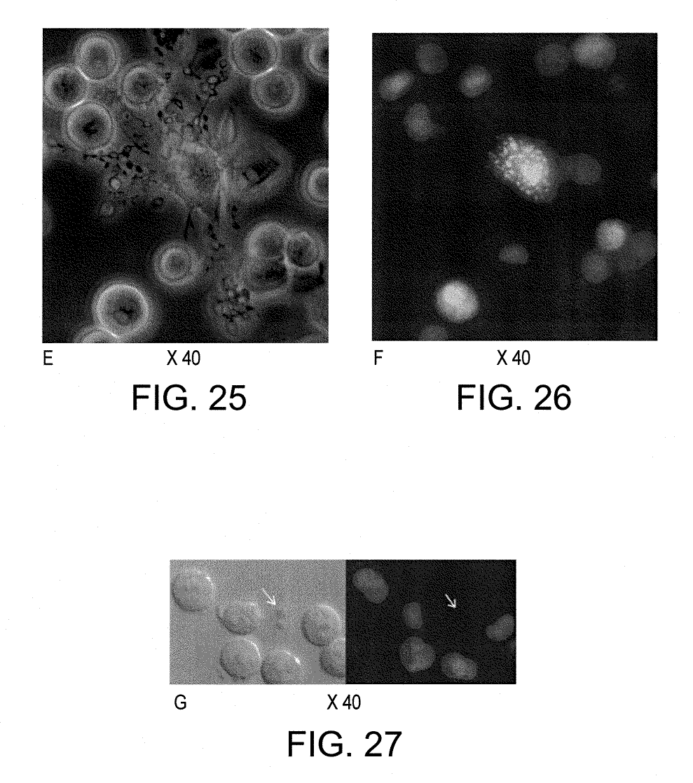

It has been well established that physiologically, platelets derive from megakaryoblasts following an apoptotic process. Since DMAT and TBB induce apoptosis in MEG-01 cells, the potential for whether CK2α inhibitors could also be thrombocytopoiesis inducers was investigated. In FIGS. 21-27, Phenotype change in MEG-01 cells following treatment with 10 μM DMAT is shown. FIG. 21 illustrates control untreated MEG-01 cells phase-contrast micrograph after 96 hours, magnification ×20, FIG. 22 shows MEG-01 cells treated with 10 μM DMAT phase-contrast micrograph, following 96 hours of treatment same magnification. FIG. 23 shows proplatelets formation, in suspension, phase-contrast micrograph, following DMAT treatment (10 μM) between 72 and 96 hours, magnification ×40. FIG. 24 shows scanning electron microscopy micrograph of MEG-01 cells, magnification ×7500, voltage 15 kV. FIG. 25 illustrates proplatelets formation on fibronectin coat, phase-contrast micrograph, following DMAT treatment (10 μM) between 72 and 96 hours of treatment, magnification ×40. FIG. 26 shows DAPI staining micrograph of proplatelets bearing MEG-01 megakaryocyte following DMAT treatment (10 μM) between 72 and 96 hours of treatment, magnification ×40. FIG. 27 shows platelets-like particles identified as anucleated cells with DAPI staining, magnification ×40 following DMAT (10 μM treatment) at 72 to 96 hours.

The data shown in FIGS. 21-27 demonstrate that CK2α inhibition result in thrombocytopoiesis. As early as from the second day DMAT induced MEG-01 cells to form proplatelet extensions. This process was dramatically enhanced following four days of treatment (FIGS. 21-26). The megakaryocytes undergoing thrombocytopoiesis showed apoptotic features, DNA condensation and fragmentation (FIGS. 23 and 25). Following explosive fragmentation, long filaments with beaded ends (proplatelets) are formed. Similar results were observed with MEG-01 in suspension (FIG. 25) and MEG-01 cells grown on fibronectin (FIG. 23). FIG. 26 shows scanning electron microscopy (SEM) of MEG-01 cells treated with 10 μM DMAT for 4 days. Pseudopodia and proplatelets formation, as well as blebbing can be observed at the surface of these cells. Platelets are expelled out from the proplatelets, and the fragmented nucleus slowly is extruded. The proplatelets do not stain positive with DAPI (FIG. 27, white arrow), showing that the beaded ends indeed will become anucleated cells. Together the data demonstrate that CK2α inhibition induces apoptosis of MEG-01 cells, which in turn result in the release of platelet-like particles.

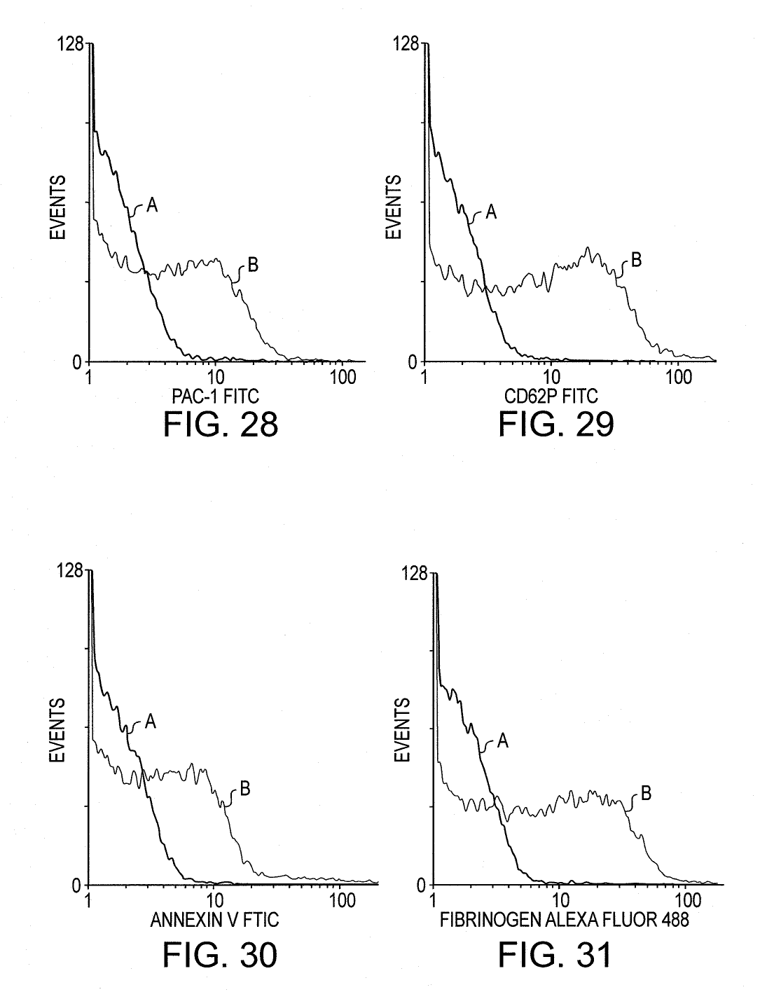

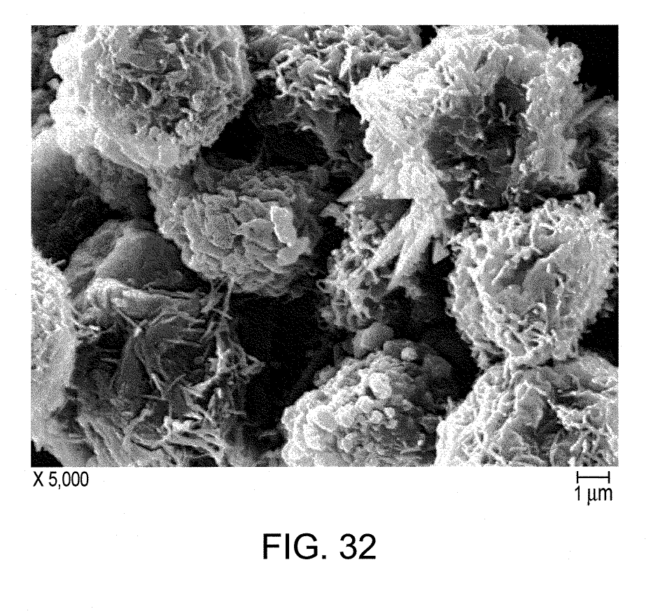

“Platelets in a dish”. The next step was to verify if the platelet-like particles released following treatment with DMAT in culture, are indeed platelets and are functional. Several functional studies were performed and the results are reported in FIGS. 28-31. In FIGS. 28-31, platelets from MEG-01 cells obtained due to DMAT treatment, in culture, are demonstrated to be functional. MEG-01-derived platelets following treatment with DMAT (10 μM for 72) were collected and used. Platelets were activated with TRAP peptide. Controls were treated with EDTA and RGDS to prevent any artefactual activation. FIG. 28 illustrates P-Selectin exposure (CD62P-FITC) by activated platelets. FIG. 29 shows PAC-1 binding (PAC-1-FITC) by activated platelets. FIG. 30 illustrates Fibrinogen-Alexa Fluor 488 binding to activated platelets. FIG. 31 illustrates Annexin V-FITC binding to activated platelets (phosphatidylserine exposure). In all noted figures, the control platelets are represented by line A, while the results obtained with activated platelets are represented by the line B. The platelets are capable of undergoing shape change in response to agonists (human thrombin, TRAP, ADP, and PMA). Activated platelets stain positive for PAC-1 (an antibody that recognizes a specific epitope on α_{IIb}β₃ integrin, exposed only when platelets are activated) (FIG. 28). Following activation, the platelets expose P-Selectin (FIG. 29) and phosphatidylserine (FIG. 30) and bind fibrinogen (FIG. 31). Finally, following activation with 0.5 U/ml of human thrombin the platelets form a visible clot. SEM demonstrated platelets develop spiked lamelapodia appearance, aggregation and formation of a fibrin net (FIGS. 32-34). Overall, this data clearly demonstrate that treatment of MEG-01 cells with casein kinase 2 inhibitors results in proliferation arrest, maturation and release of functional platelets.

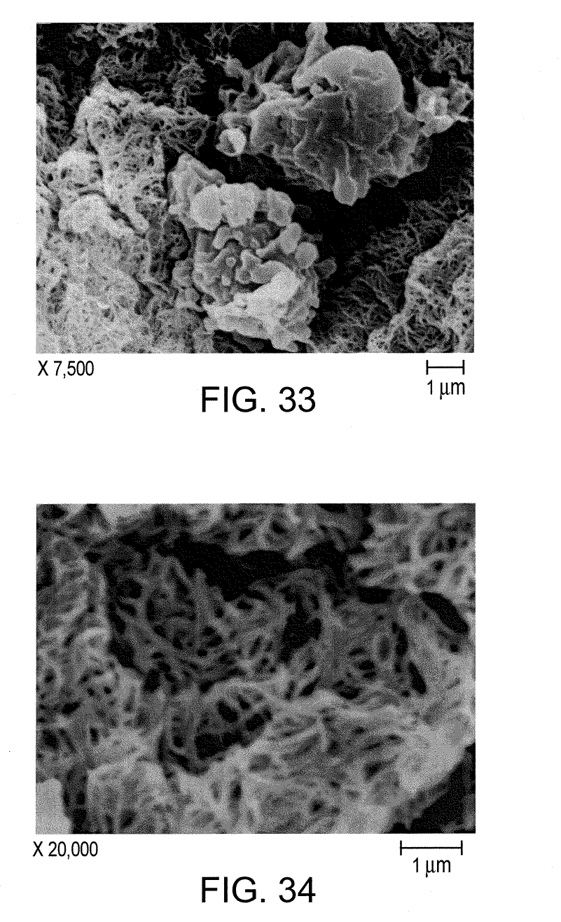

And, in FIGS. 32-34, platelets from MEG-01 cells obtained in culture, following DMAT treatment, form a fibrin clot when activated with thrombin. Platelets were harvested from MEG-01 cells grown in the presence of DMAT (10 μM at 72 hours). Human thrombin 0.5 U/ml was used as agonist. In FIG. 32, details of the fibrin clot magnification ×5,000, voltage 20 kV are shown. In FIG. 33, platelets and fibrin net detail, magnification ×7,500, voltage 20 kV are shown. And, in FIG. 34, fibrin net detail from the clot, magnification ×20,000 voltage 20 kV is shown.

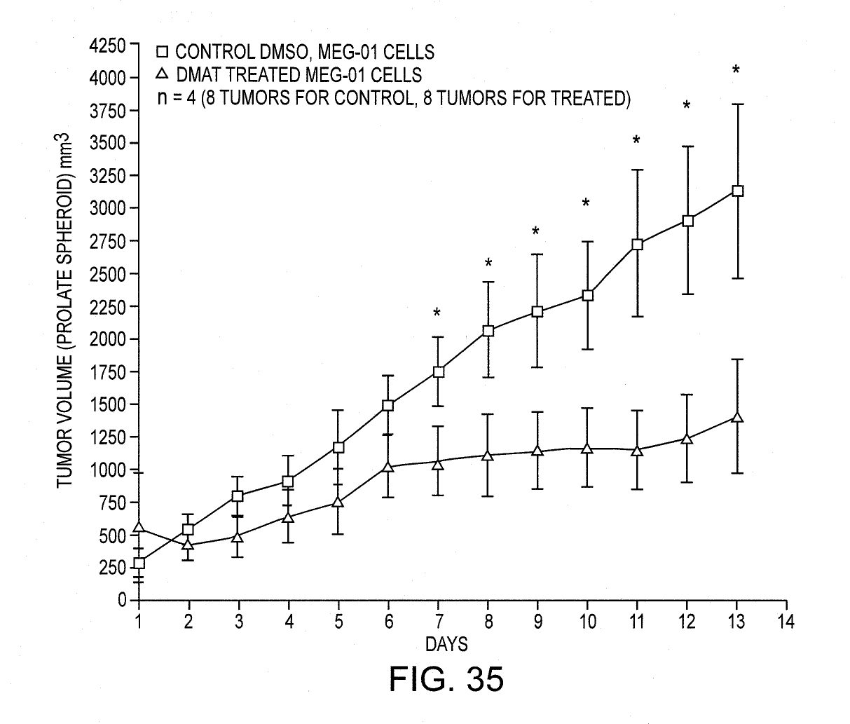

FIG. 35 is a graph of a murine xenograft treated with DMAT, compared to a control. Specifically, mice with subcutaneous tumors of MEG-01 cells were treated with DMAT 2 mg (in DMSO) per animal per day. The daily animal weight was averaged at 32.5 g at the start of the treatment. Tumors were measured daily and tumor volumes were calculated based upon a prolate spheroid model. FIG. 35 illustrates the relatively rapid growth of tumor volume of the control (squares) as compared to tumor volume of DMAT treated MEG-01 cells (triangles). FIG. 35 further illustrates that DMAT induces proliferation arrest in vivo in large tumors and tumor ablation in small tumors, the arrest being dose and time dependent.

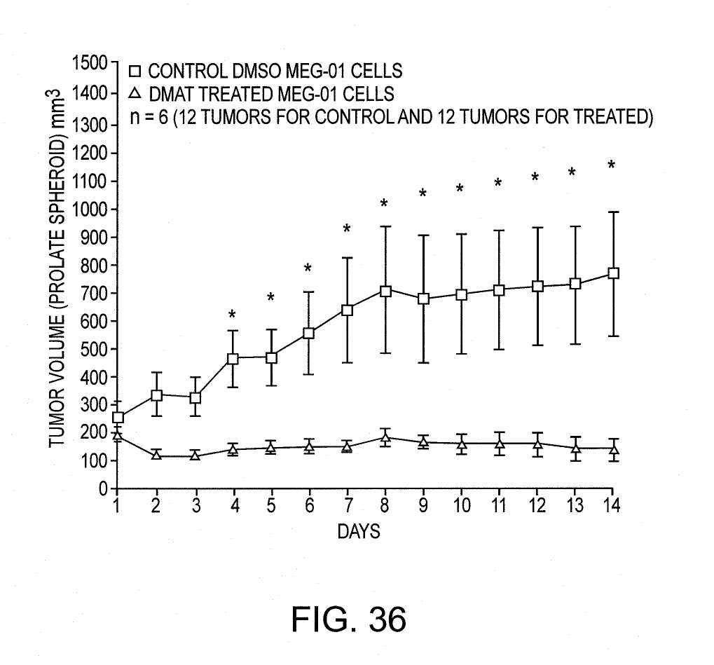

FIG. 36 is a graph of a murine xenograft treated with DMAT, compared to a control. Mice with subcutaneous tumors of MEG-01 cells were treated with DMAT 3 mg (in DMSO) per animal per day. The daily animal weight was averaged at 32.5 g at the start of treatment. Tumors were measured daily and tumor volumes were calculated based upon a prolate spheroid model. FIG. 36 illustrates the significant benefits of treatment with DMAT (triangles) as compared to the control (squares). FIG. 36 further illustrates DMAT induces proliferation arrest in vivo for large tumors and tumor ablation in small tumors, such effect being dose and time dependent.

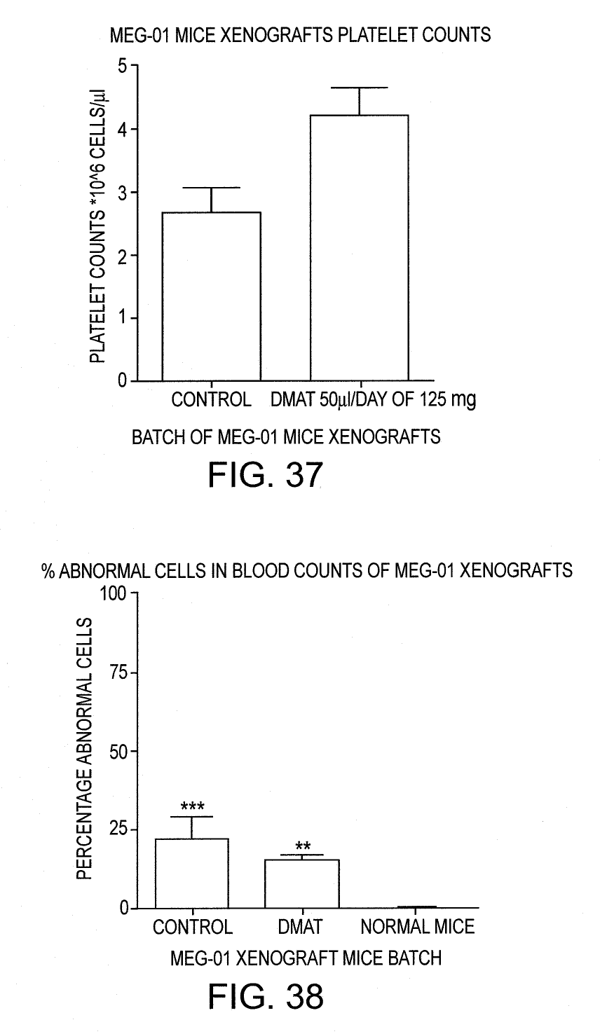

FIGS. 37 and 38 illustrate additional aspects of the MEG-01 xenografts. FIG. 37 shows an increase in platelet counts in a batch of MEG-01 mice xenografts treated with DMAT 50 μl/day of 125 mg compared to a control. FIG. 38 illustrates the percentage of abnormal cells in blood counts of MEG-01 xenografts of a control, DMAT treated MEG-01 cells, and normal mice. FIGS. 37 and 38 demonstrate that MEG-01 xenograft mice have abnormally high platelet counts, i.e. both with regard to a DMSO control and DMAT-treated specimens. And, the figures reveal that the treated cells have higher platelet counts, the MEG-01 xenograft mice have abnormal cells in blood counts, and the control has higher percentages of abnormal cells.

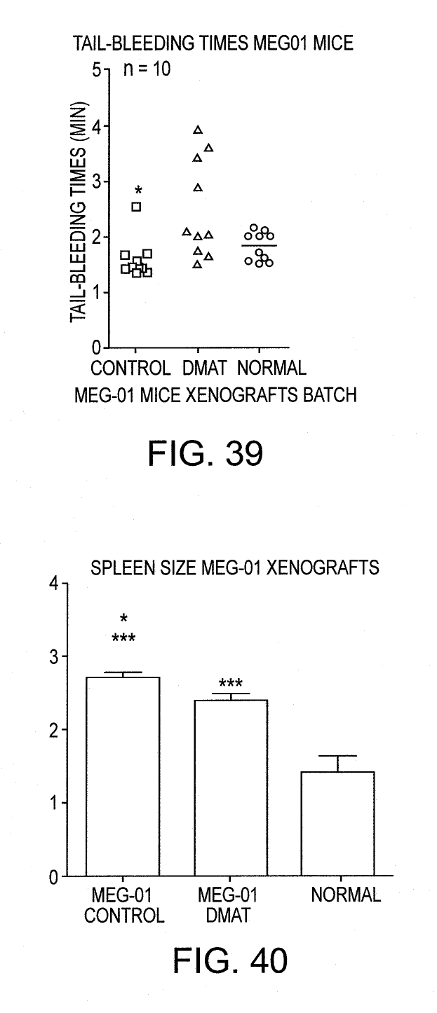

FIG. 39 is a graph illustrating the results of tail bleeding times in MEG-01 xenograft mice. MEG-01 tumors produce platelets in vivo. This is similar as to what happens in acute myelogenous leukemia (CML). This data illustrates that tail bleeding times in the xenograft mice (triangles) are in certain instances, longer than tail bleeding times in normal mice (circles) or control mice (squares). This is believed to be a result of DMAT being a CK2 inhibitor (such as for example, like heparin). Human platelets do not respond to mouse thrombin and so, this fact may affect tail bleeding times.

FIG. 40 is a graph illustrating comparative spleen sizes between MEG-01 xenografts (treated with DMAT and a control) and that of normal mice. Spleen size differences between the treated and the control may be due to the fact that MEG-01 tumors produce platelets and circulating blasts in vivo and the controls have larger tumors than the treated and more abnormal cells. This same observation is also valid for the treated xenograft only. This phenomena is similar with what happens in chronic myelogenous leukemia (CML).

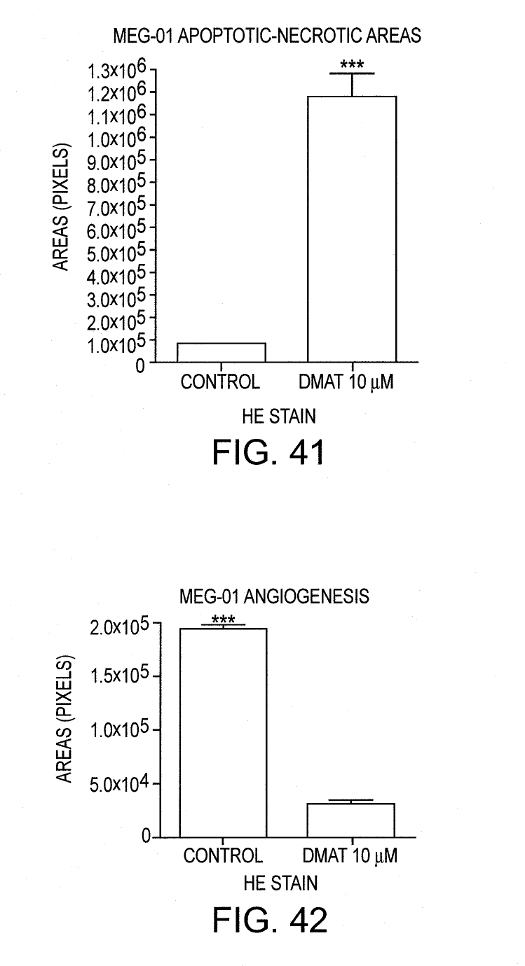

FIG. 41 illustrates apoptotic-necrotic areas in MEG-01 tumors identified from the histological stains hematoxylin and eosin. FIG. 41 shows the significantly greater areas of apoptosis and necrosis of a DMAT treated (10 μM) MEG-01 cell line as compared to a control. FIG. 42 illustrates an area of angiogenesis of a DMAT treated (10 μM) MEG-01 cell line as compared to a control. Angiogenesis refers to blood vessel formation which usually accompanies the growth of malignant tissue.

### Investigation of DMAT Toxicity

In another study, a male athymic nude mice nu/nu was treated with injection, subcutaneous in the neck with 200 mg/kg per day DMAT (in DMSO) for 2 weeks. At the injection site the skin was irritated and swollen like a cyst. The animal was exhibiting generally normal behavior (eating, drinking) except showing increased irritability. The liver appeared to have increased in size. Samples of injection site tissue, liver, spleen, brain and bone (for bone marrow) were collected for further analysis.

### Investigation of Effect of Preferred Inhibitors Upon Breast Cancer (MCF-7)

In this study, a cell line designated as MCF-7 was obtained. MCF-7 is a breast cancer cell line established from the mammary gland of a 69 year old woman. It is an adenocarcinoma derived from pleural effusion (metastic site). MCF-7 are differentiated mammary epithelium cells that express estrogen receptor. MCF-7 cells express oncogenes (WNT7B and Tx-4) and are sensitive to TNF alpha, which inhibits their growth. MCF-7 growth in vivo is hormone dependent (estradiol). MCF-7 cells produce insulin-like growth factor binding proteins (IGFBP). IGFBP secretion from MCF-7 can be modulated by treatment with anti-estrogen.

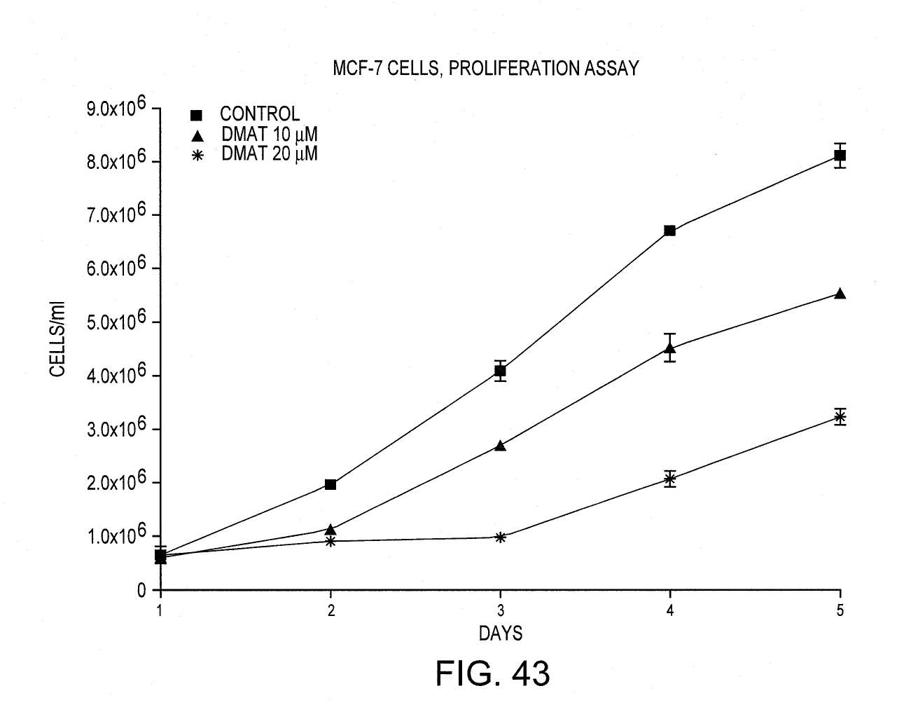

FIG. 43 illustrates a proliferation assay in vitro of MCF-7 cells. Various cell counts were performed over a period of five days. The control (squares) exhibited the highest number of cells. The MCF-7 cells treated with 20 μM DMAT exhibited the lowest cell counts. The MCF-7 cells treated with 10 μM DMAT exhibited cell counts between the control and the MCF-7 cells treated with 20 μM at 24 hours (FIG. 46).

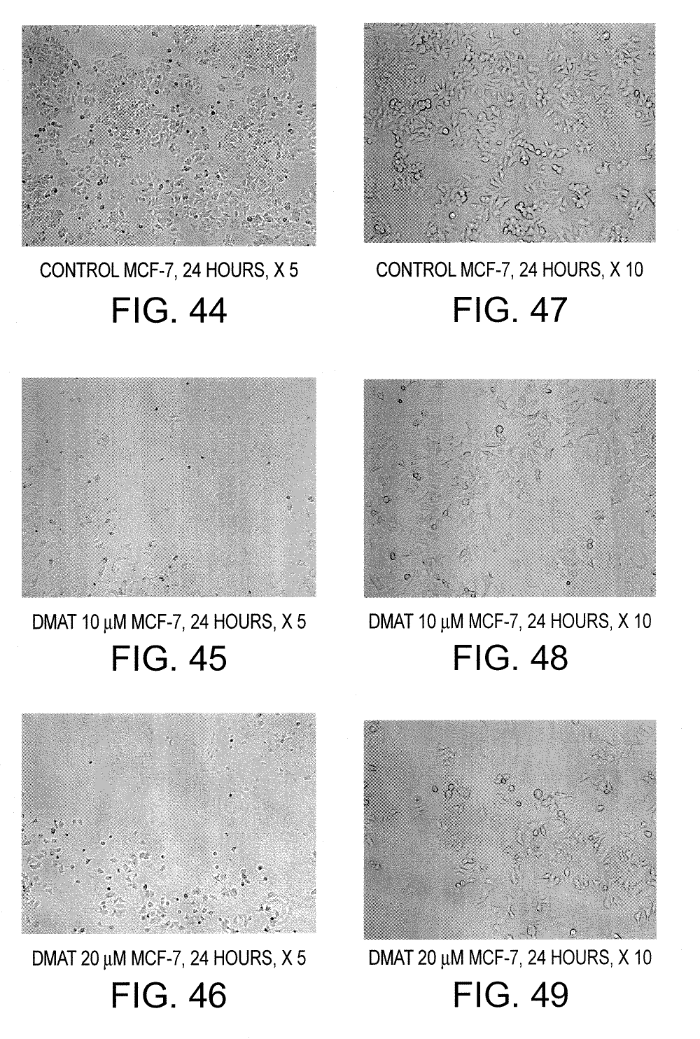

FIGS. 44-49 are photographs illustrating the arrest of MCF-7 proliferation, in vitro. FIGS. 44-46 are photographs at 5× showing a control of MCF-7 at 24 hours (FIG. 44), MCF-7 treated with DMAT 10 μM at 24 hours (FIG. 45), and MCF-7 treated with DMAT 20 μM at 24 hours (FIG. 46). FIGS. 47-49 are photographs at 10× showing a control of MCF-7 at 24 hours (FIG. 47), MCF-7 treated with DMAT 10 μM at 24 hours (FIG. 48), and MCF-7 treated with DMAT 20 μM at 24 hours (FIG. 49).

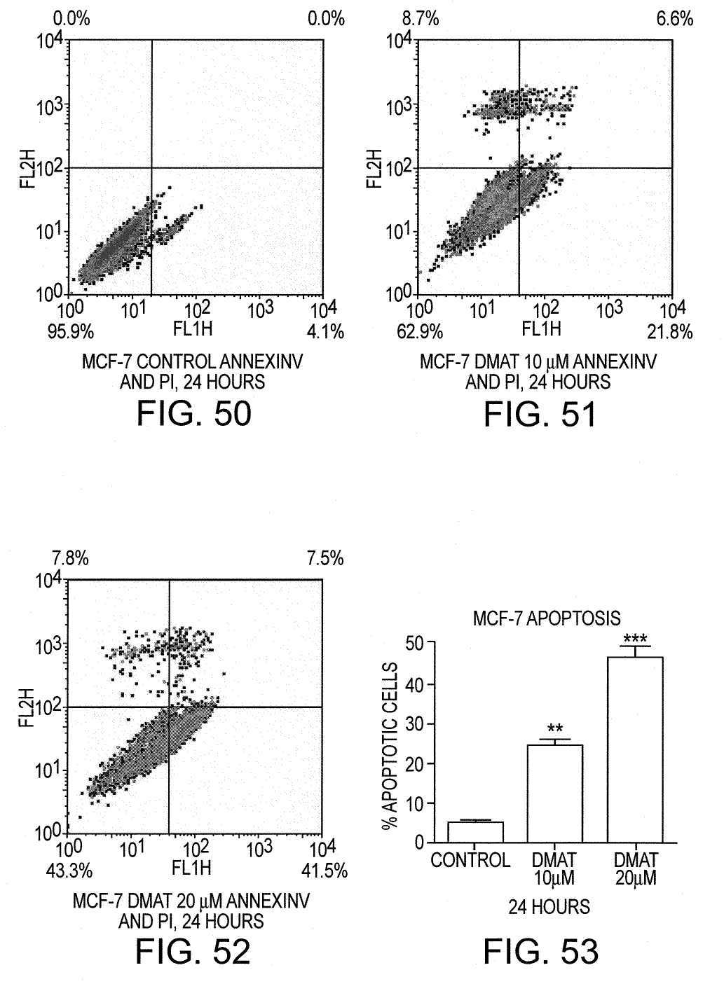

FIGS. 50-53 are graphs illustrating assessment of the effect of the preferred inhibitor DMAT on MCF-7 cells in vitro. And so, using an assay employing Annexin V, assessment of both apoptosis and necrotic levels was made. FIG. 50 demonstrates that following 24 hours incubation in the absence of CK2 inhibitors, 41% of the control untreated cells are apoptotic while 0% are necrotic (FIG. 50). Following 24 hours treatment with 10 μM DMAT, the level of apoptotic cells significantly increased to 28.4% while the level of necrotic cells remained low at 8.7% (FIG. 51). In another sample following treatment with 20 μM DMAT, the level of apoptotic cells increased to 49.0% while the level of necrotic cells remained at a very low value of 7.8% (FIG. 52). FIG. 53 is a comparative graph illustrating the percentage of apoptotic cells in the control and the MCF-7 DMAT 10 μM sample and the MCF-7 DMAT 20 μM sample.

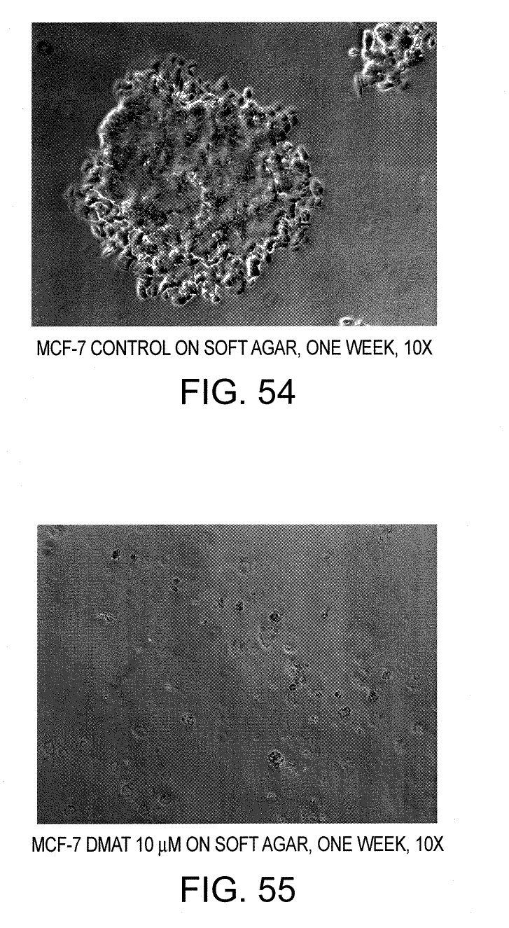

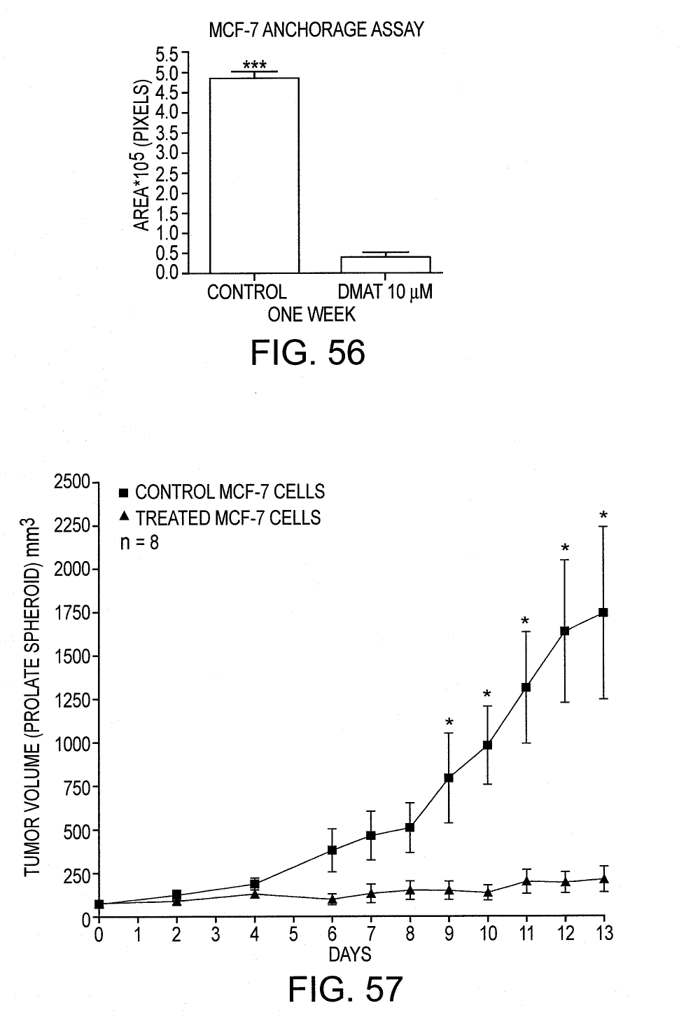

FIGS. 54-55 are photographs illustrating MCF-7 anchorage independence. FIG. 54 illustrates MCF-7 control on soft agar after one week, at 10×. FIG. 55 illustrates MCF-7 treated with DMAT 10 μM on soft agar after one week, at 10×. FIG. 56 is a comparative graph showing relative area of the control and the noted MCF-7 treated cells.

FIG. 57 is a graph of mice injected with the MCF-7 cell line, i.e. a murine xenograft model, illustrating changes in tumor volume over a period of thirteen days. A control (squares) exhibited significant increase in volume, i.e. from about 50 mm³ to about 1750 mm³. In contrast, the MCF-7 cells treated with DMAT at 10 mg/kg (in DMSO) per day, exhibited remarkably stable size and nearly no increase over the 13 day period. In this study, mice were injected with subcutaneous tumors of MCF-7 cells. Tumors were measured daily and volumes were calculated based on a prolate spheroid model. Animal weight was averaged at 25 g at the beginning of treatment. This investigation demonstrates that DMAT induces proliferation arrest in vivo in large tumors, and tumor ablation in small tumors, and is dose and time dependent.

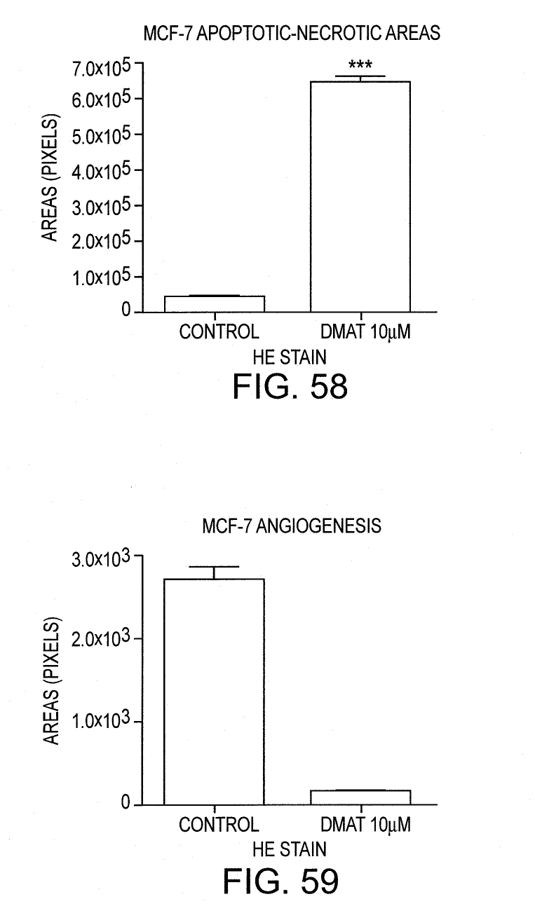

FIG. 58 illustrates relative areas of apoptotic-necrotic MCF-7 cells treated with DMAT 10 μM. Angiogenesis in MCF-7 cells treated with DMAT 10 μM is shown in FIG. 59 compared to controls. As previously explained, the cells were stained with hematoxylin and eosin, which are two known histological stains.

### Investigation of Effect of Preferred Inhibitors Upon Colon Cancer (SW-480)

In this study, a cell line designated as SW-480 was obtained. SW-480 was established from a 50 year old male. SW-480 are colon epithelial cells from colorectal adenocarcinoma, tumor stage: Dukes' type B. SW-480 cells produce carcinoembryonic antigen (CEA), keratin, transforming growth factor beta. SW-480 cells exhibit epithelial growth factor receptor (EGF). SW-480 cells can be infected with Human immunodeficiency virus 1.

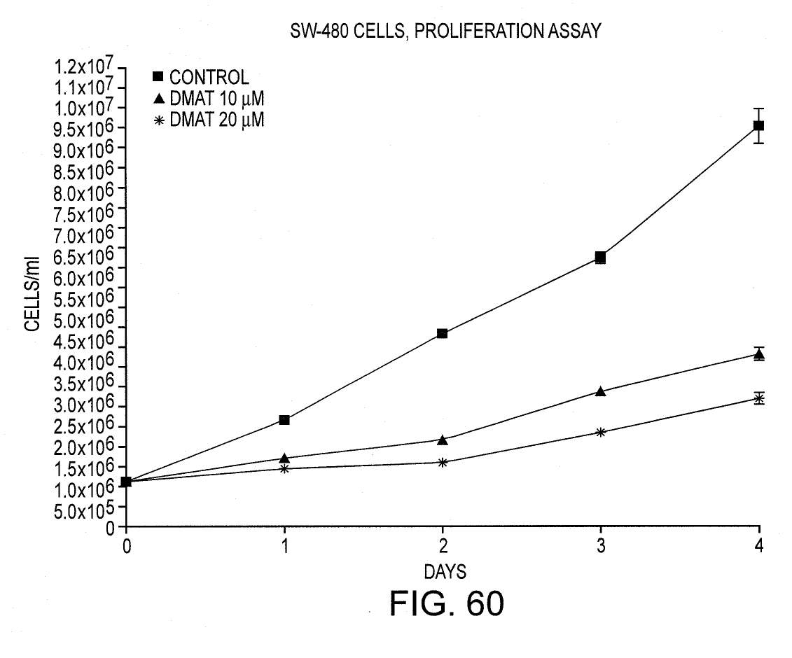

FIG. 60 illustrates a proliferation assay in vitro of SW-480 cells. Various cell counts were performed over a period of four days. The control (squares) exhibited the highest number of cells. The SW-480 cells treated with 20 μM DMAT exhibited the lowest cell counts. The SW-480 cells treated with 10 μM DMAT exhibited cell counts between the control and the SW-480 cells treated with 20 μM.

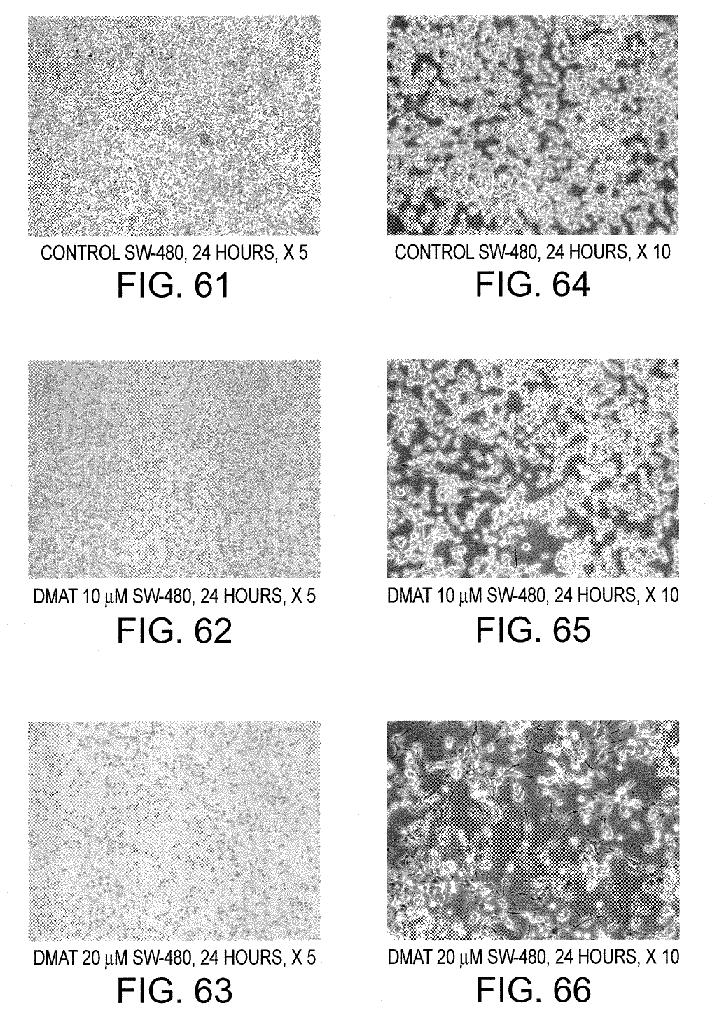

FIGS. 61-66 are photographs illustrating the arrest of SW-480 proliferation, in vitro. FIGS. 61-63 are photographs at 5× showing a control of SW-480 at 24 hours (FIG. 61), SW-480 treated with DMAT 10 μM at 24 hours (FIG. 62), and SW-480 treated with DMAT 20 μM at 24 hours (FIG. 63). FIGS. 64-66 are photographs at 10× showing a control of SW-480 at 24 hours (FIG. 64), SW-480 treated with DMAT 10 μM at 24 hours (FIG. 65), and SW-480 treated with DMAT 20 μM at 24 hours (FIG. 66).

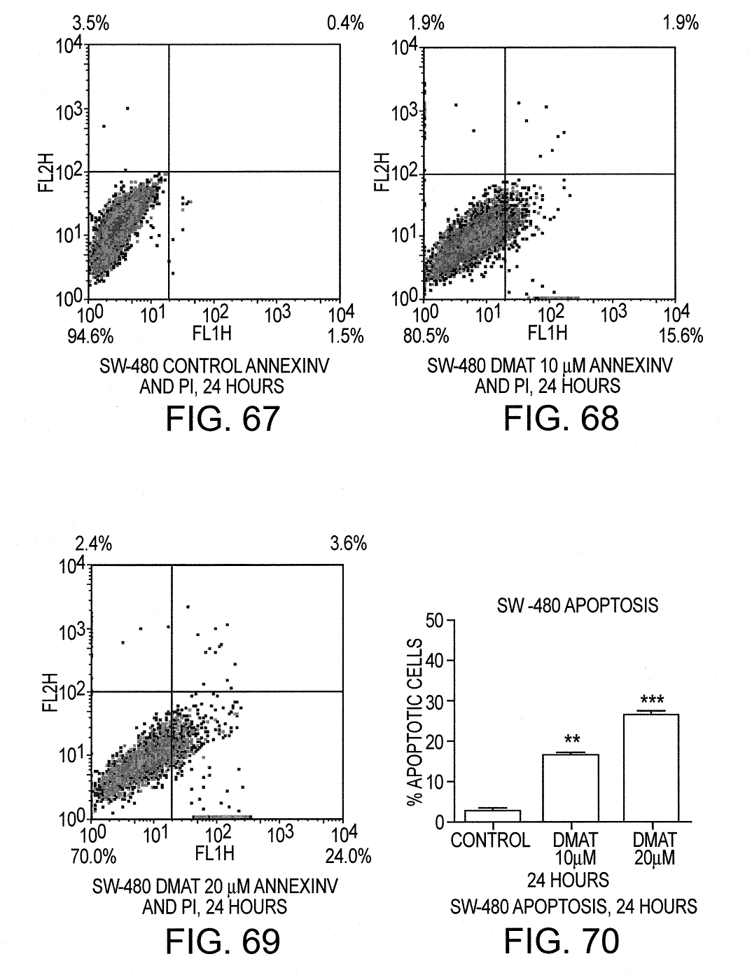

FIGS. 67-70 are graphs illustrating assessment of the effect of the preferred inhibitor DMAT on SW-480 cells in vitro. Using an assay employing Annexin V, assessment of both apoptosis and necrotic levels was made. FIG. 67 demonstrates that the following 24 hours incubation in the absence of CK2 inhibitors, 1.9% of the control untreated cells are apoptotic while 3.5% are necrotic. Following 24 hours treatment with 10 μM DMAT, the level of apoptotic cells significantly increased to 17.5% while the level of necrotic cells remained very low at 1.9% (FIG. 68). In another sample following treatment with 20 μM DMAT, the level of apoptotic cells increased to 27.6% while the level of necrotic cells remained at only 2.4% (FIG. 69). FIG. 70 is a comparative graph illustrating the percentage of apoptotic cells in the control and the SW-480 DMAT 10 μM sample and the SW-480 DMAT 20 μM sample.

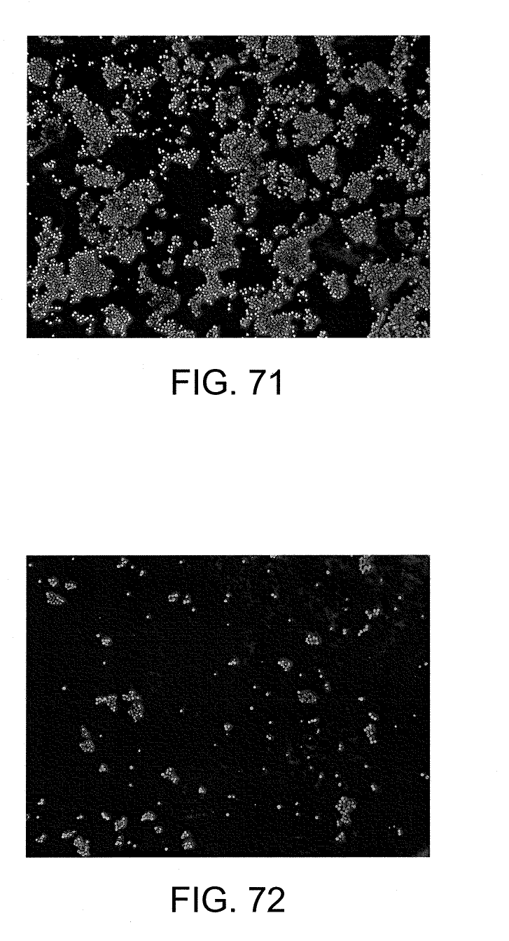

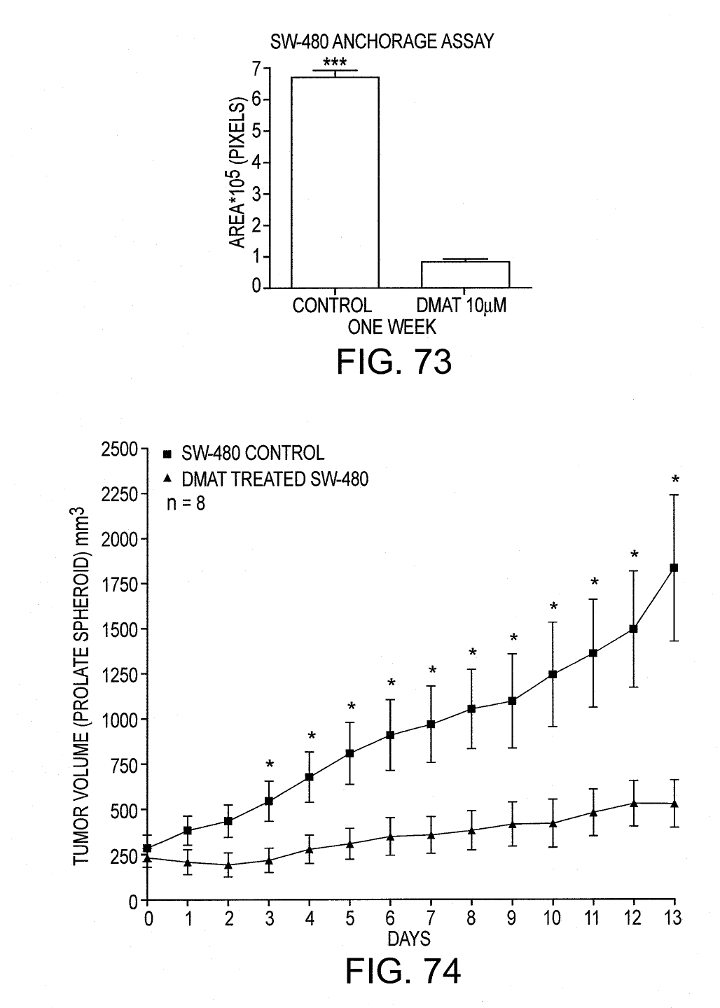

FIGS. 71-72 are photographs illustrating SW-480 anchorage independence. FIG. 71 illustrates SW-480 control on soft agar after one week, at 10×. FIG. 72 illustrates SW-480 treated with DMAT 10 μM on soft agar after one week, at 10×. FIG. 73 is a comparative graph showing relative area of the control and the noted SW-480 treated cells.

FIG. 74 is a graph of a murine xenograft model, i.e. mice injected with the SW-480 cell line, illustrating changes in tumor volume over a period of thirteen days. A control (squares) exhibited a dramatic increase in tumor volume, i.e. from about 260 mm³ to about 1800 mm³. In contrast, the SW-480 cells treated with DMAT at 40 mg/kg (in DMSO) per day, exhibited only a minor increase in tumor volume. In this study, mice were injected with subcutaneous tumors of SW-480 cells. Tumors were measured daily and volumes calculated based upon a prolate spheroid model. Animal weight was averaged at 35 g at the start of treatment. This investigation demonstrates that DMAT induces proliferation arrest in vivo in large tumors, and tumor ablation in small tumors and is dose and time dependent.

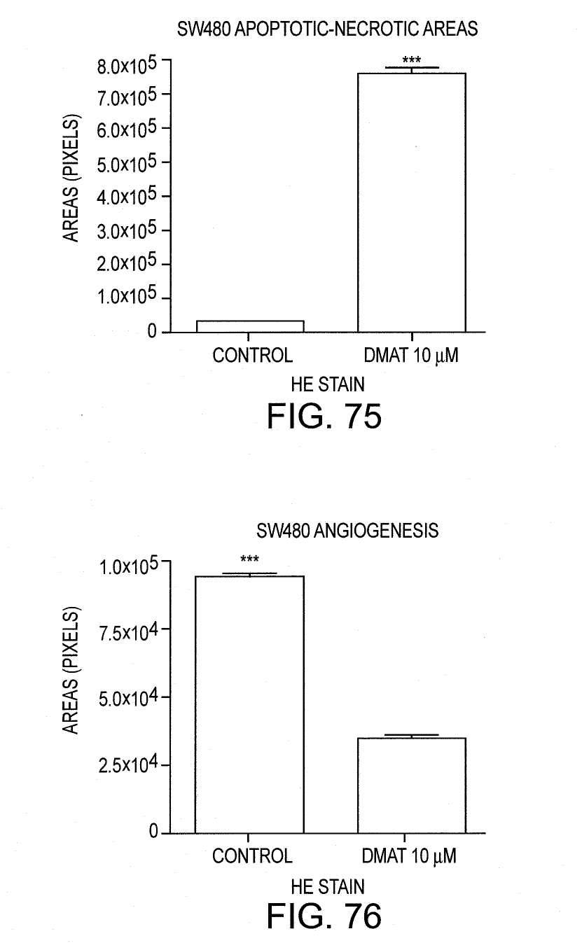

FIG. 75 illustrates relative areas of apoptotic-necrotic SW-480 cells treated with DMAT 10 μM. FIG. 76 illustrates angiogenesis in SW-480 cells treated with DMAT 10 μM as compared to a control. As previously explained, cells were stained with hematoxylin and eosin.

### Investigation of Effect of Preferred Inhibitors Upon Melanoma (WM-164)

In this study, a cell line designated as WM-164 was obtained. WM-164 was established from a 21 year old male with nodular melanoma in vertical growth phase. WM-164 are skin melanocytes. WM-164 exhibit spontaneous metastasis into liver and lung.

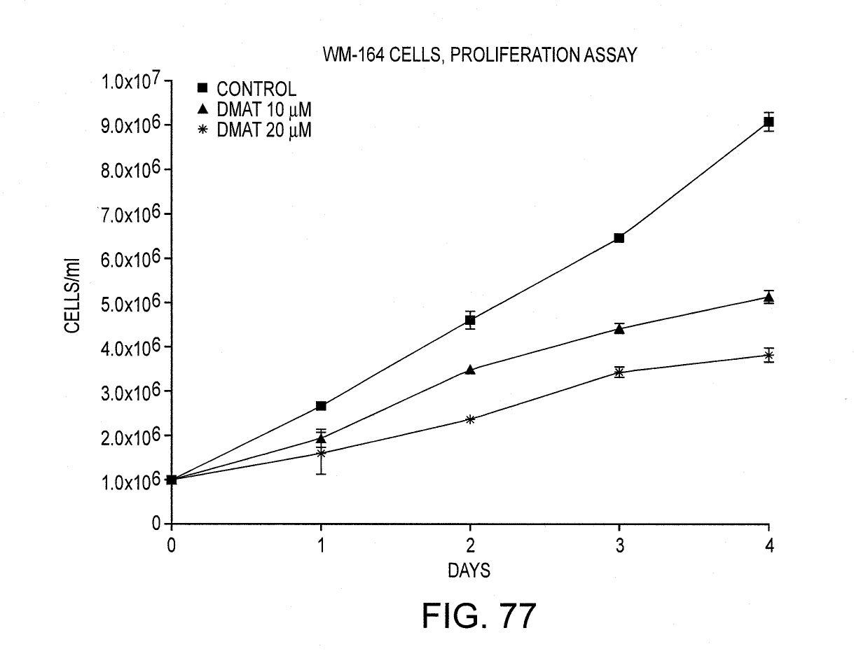

FIG. 77 illustrates a proliferation assay in vitro of WM-164 cells. Cell counts were performed over a period of four days. The control (squares) exhibited the highest number of cells. The WM-164 cells treated with 20 μM DMAT exhibited the lowest cell counts. The WM-164 cells treated with 10 μM DMAT exhibited cell counts between those of the control and those of the WM-164 cells treated with 20 μM.

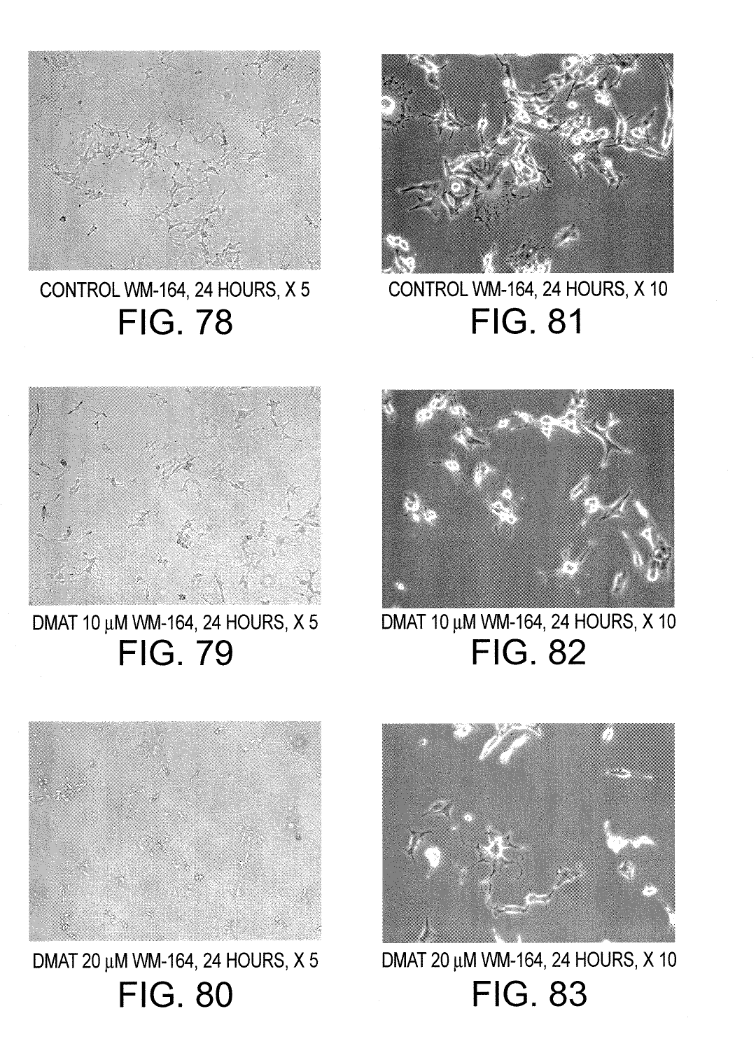

FIGS. 78-83 are photographs showing the arrest of WM-164 proliferation, in vitro. FIGS. 78-80 are photographs at 5× showing a control of WM-164 at 24 hours (FIG. 78), WM-164 treated with DMAT 10 μM at 24 hours (FIG. 79), and WM-164 treated with DMAT 20 μM at 24 hours (FIG. 80). FIGS. 81-83 are photographs at 10× showing a control of WM-164 at 24 hours (FIG. 81), WM-164 treated with DMAT 10 μM at 24 hours (FIG. 82) and WM-164 treated with DMAT 20 μM at 24 hours (FIG. 83).

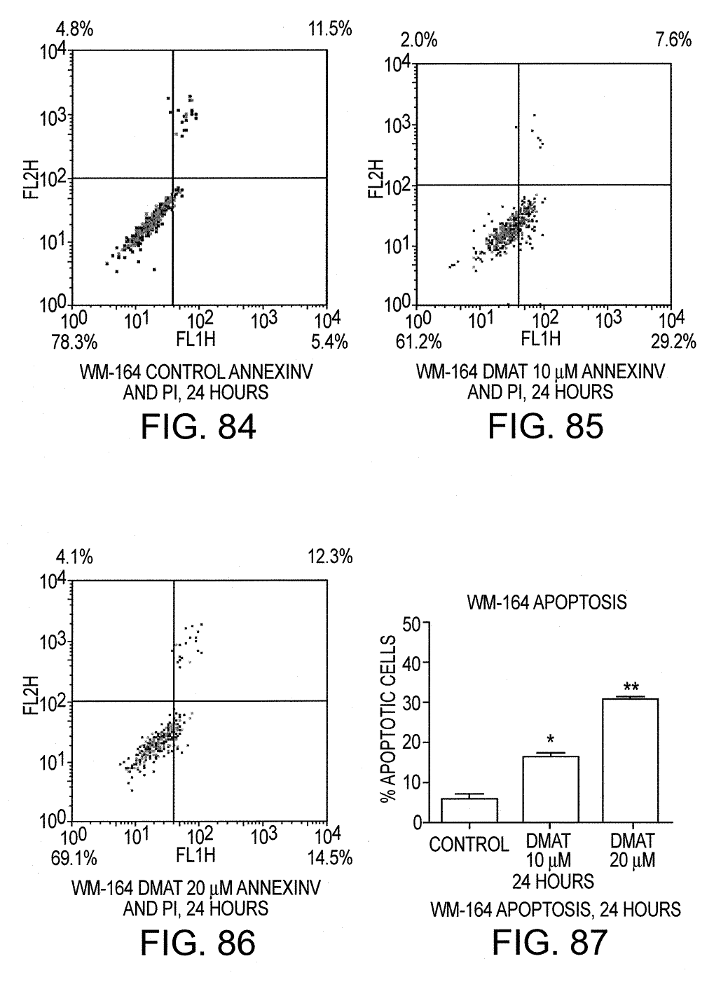

FIGS. 84-86 are graphs illustrating assessment of the effect of the preferred inhibitor DMAT on WM-164 cells, in vitro. Using an assay employing Annexin V, assessment of both apoptosis and necrotic levels was made. FIG. 84 demonstrates that following 24 hours incubation in the absence of CK2 inhibitors, 16.9% of the control untreated cells are apoptotic while 4.8% are necrotic. Following 24 hours treatment with 10 μM DMAT, the level of apoptotic cells increased to 36.8% while the level of necrotic cells remained low at 2.0% (FIG. 85). In another sample following treatment with 20 μM DMAT, the level of apoptic cells was 26.8% while the level of necrotic cells was 4.1%.

FIG. 87 is a comparative graph illustrating the percentage of apoptotic cells in the control and the WM-164 DMAT 10 μM sample and the WM-164 DMAT 20 μM sample.

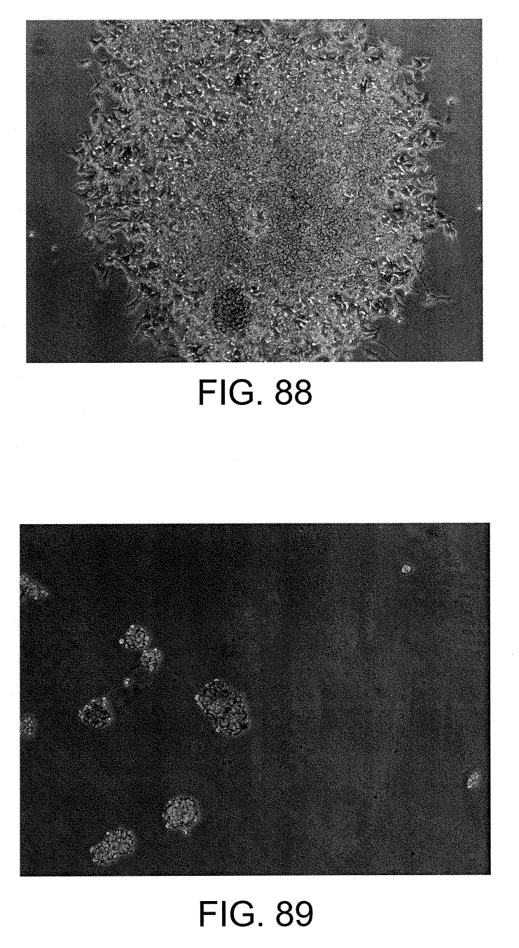

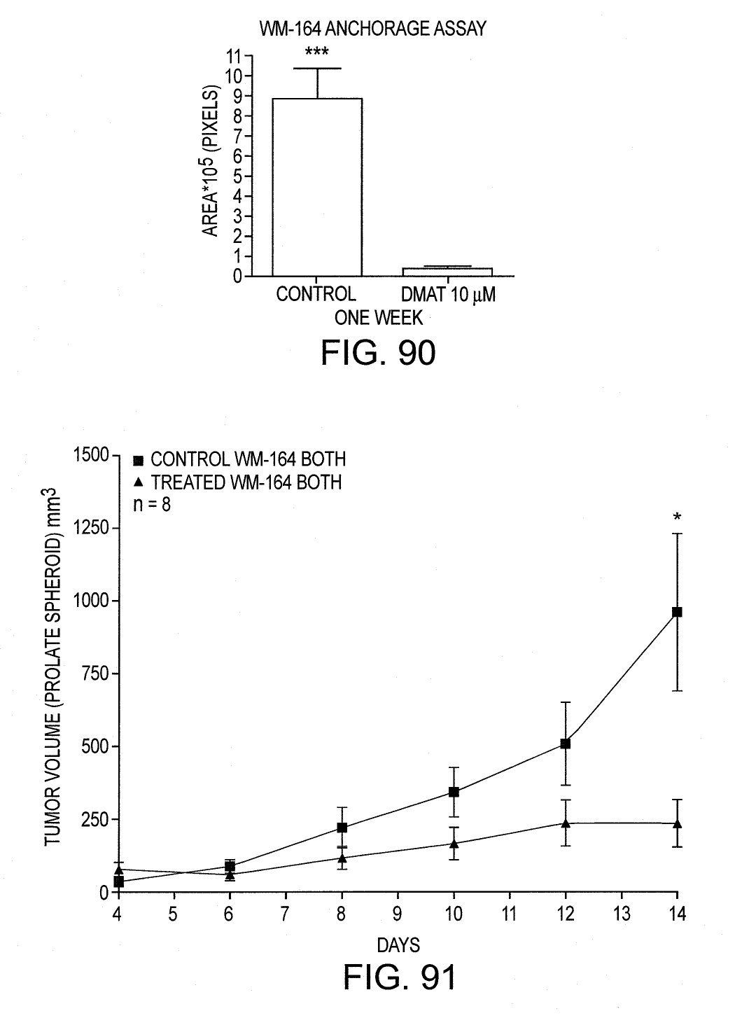

FIGS. 88-89 are photographs illustrating WM-164 anchorage independence. FIG. 88 shows WM-164 control on soft agar after one week, at 10×. FIG. 89 shows WM-164 treated with DMAT 10 μM on soft agar after one week, at 10×. FIG. 90 is a comparative graph showing relative area of the control and the noted WM-164 treated cells.

FIG. 91 is a graph of a murine xenograft model, i.e. mice injected with the WM-164 cell line, illustrating changes in tumor volume over a period of fourteen days. A control (squares) exhibited a significant increase in tumor volume, i.e. from about 50 mm³ to about 1000 mm³ in that time period. In sharp contrast, the WM-164 cells treated with DMAT at 10 mg/kg (in DMSO) per day, exhibited only a slight increase in tumor volume. In this study, mice were injected with subcutaneous tumors of WM-164 cells. Tumors were measured daily and volumes calculated based upon a prolate spheroid model. Animal weight was averaged at about 35 g at the beginning of the study. This study demonstrates that DMAT induces proliferation arrest in vivo for large tumors, and tumor ablation for small tumors, and is also dose and time dependent.

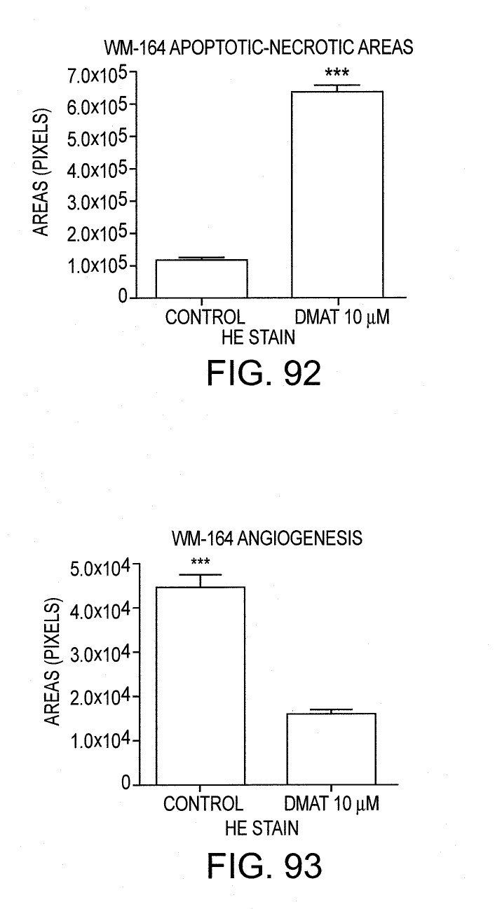

FIG. 92 illustrates relative areas of apoptotic-necrotic WM-164 cells treated with DMAT 10 μM. FIG. 93 illustrates angiogenesis in WM-164 cells treated with DMAT 10 μM as compared to a control. As previously explained, cells were stained with hematoxylin and eosin.

### Investigation of Effect of Preferred Inhibitors Upon Renal Cell Carcinoma (ACHN)

In this study, a cell line designated as ACHN was obtained. ACHN are cancerous renal cells.

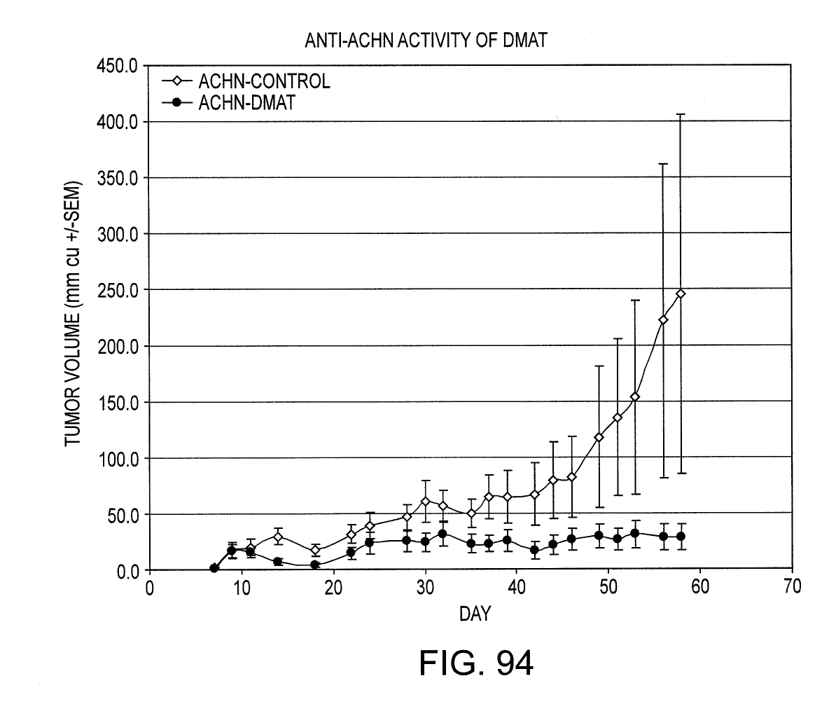

FIG. 94 illustrates the effect of administration of DMAT as compared to a control over a period of 59 days. FIG. 94 is a graph of a murine xenograft treated with DMAT, compared to a control. Mice with tumors of ACHN cells were treated with DMAT at effective dosage levels (in DMSO) per animal per day. The daily animal weight was averaged at 32.5 g at the start of treatment. Tumors were measured daily and tumor volumes were calculated based upon a prolate spheroid model. FIG. 94 illustrates the significant benefits of treatment with DMAT (circles) as compared to the control (squares). FIG. 94 further illustrates DMAT induces proliferation arrest in vivo for large tumors and tumor ablation in small tumors, such effect being dose and time dependent. The differences in tumor volume between control cells and those treated with DMAT, as described herein, are striking.

### Investigation of Effect of Preferred Inhibitors Upon Cancerous Bladder Cells (HT1376)

In this study, a cell line designated as HT1376 was obtained. HT1376 are cancerous bladder cells.

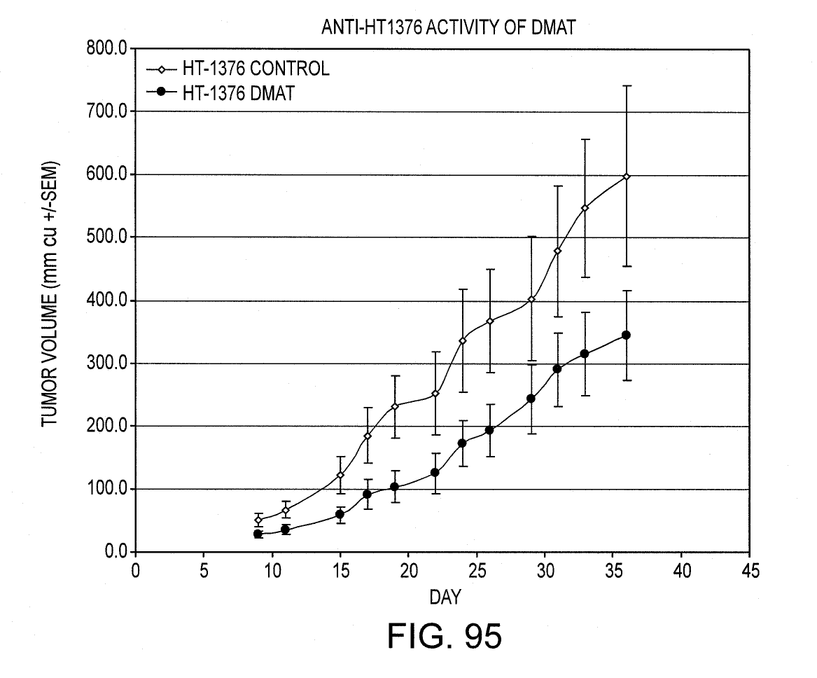

FIG. 95 illustrates the effect of administration of DMAT s compared to a control over a time period of 36 days. FIG. 95 is a graph of a murine xenograft treated with DMAT, compared to a control. Mice with tumors of HT1376 cells were treated with DMAT at effective dosage levels (in DMSO) per animal per day. The daily animal weight was averaged at 32.5 g at the start of treatment. Tumors were measured daily and tumor volumes were calculated based upon a prolate spheroid model. FIG. 95 illustrates the significant benefits of treatment with DMAT (circles) as compared to the control (squares). Although the tumor size increased for HT1376 cells treated with DMAT, the tumor volume remained approximately one-half of the size as that associated with the untreated HT1376 cells.

### Investigation of Effect of Preferred Inhibitors Upon Glioblastoma (U-87)

In this study, a cell line designated as U-87 was obtained. U-87 are cancerous brain cells.

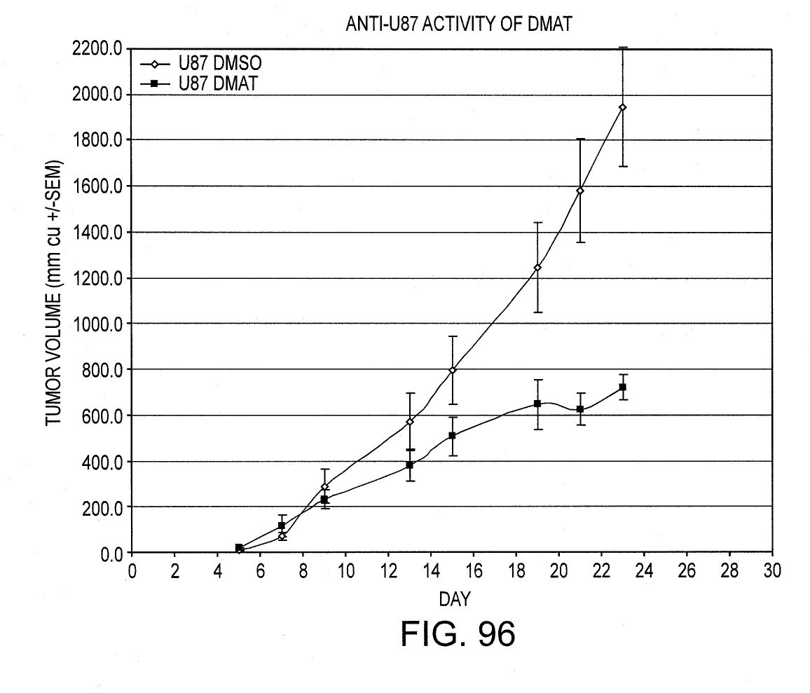

FIG. 96 illustrates the effect of administration of DMAT as compared to a control, i.e. DMSO, for a period of approximately 24 days. FIG. 96 is a graph of a murine xenograft treated with DMAT, compared to a control. Mice with tumors of U-87 cells were treated with DMAT at effective dosage levels (in DMSO) per animal per day. The daily animal weight was averaged at 32.5 g at the start of treatment. Tumors were measured daily and tumor volumes were calculated based upon a prolate spheroid model. FIG. 96 illustrates the significant benefits of treatment with DMAT (squares) as compared to the control (diamonds). FIG. 96 further illustrates DMAT induces proliferation arrest in vivo for large tumors and tumor ablation in small tumors, such effect being dose and time dependent. Again, the U-87 brain cells treated with DMAT exhibited significantly less cancerous growth than the untreated control cells.

The foregoing data demonstrate for the first time that CK2α inhibition induces malignant MEG-01 megakaryoblasts maturation and enhances functional platelet progeny release. Interestingly, it was discovered that CK2α inhibition with DMAT and TBB, induces proliferation arrest and apoptosis, without being cytotoxic. Proliferation arrest as well as apoptosis was correlated with length and amount of treatment. DMAT, which is a better inhibitor than TBB, had effect at concentrations as little as 5 and 10 μM. Due to the importance of protein kinases in malignant processes, this study can be considered to have consequences for future therapeutic interest.

A striking observation that results from the present investigation is that CK2α inhibition with DMAT and TBB induced thrombocytopoiesis. The megakaryocytes undergoing thrombocytopoiesis showed apoptotic features, as DNA condensation and fragmentation, blebbing and phosphatidylserine exposure. Mature megakaryocytes start to bleb and form pseudopodia. Following explosive fragmentation, long filaments with beaded ends (proplatelets) are formed. The proplatelets do not stain positive in DAPI, demonstrating that the beaded ends indeed will become platelets. Platelets are expelled out from the proplatelets, and the fragmented nucleus slowly is extruded. Thrombocytopoiesis process occurred in cells bound to fibronectin matrix as well on cells in suspension.

The thrombocytopoiesis process observed in the present study follows the maturation and differentiation process of MEG-01 megakaryoblasts. This differentiation is similar to the effect observed with phorbol ester (PMA), however, CK2α inhibitors are not cytotoxic, whereas PMA is a potent tumorigenic substance as well as a powerful platelet activator. It is contemplated that maturation of MEG-01 cells is a result of proliferation arrest that makes incomplete repeated cell cycle to enter into endomitosis, probably due to the action of CK2 on the cell cycle. This hypothesis is strongly supported by the preponderant nuclear localization of CK2α in malignant cells as compared to its localization in normal cells. This maturation process was assessed by flow cytometry. α_{IIb}β₃ integrin increase in expression is correlated with increase in size and differentiation of megakaryoblasts. Increase in DNA content and cell size following incubation of the cells with the inhibitors, demonstrates that MEG-01 cells mature due to CK2α inhibition.

Anchorage independence assays in soft agar show that CK2α inhibition with DMAT represses the malignant nature of MEG-01 cells. These results suggest that adherence pathways controlled by CK2α may also be involved in the process. BCR/ABL was found to be involved in the malignant transformation of Ph+ cells, but its inhibition is not sufficient to suppress anchorage independence of such cells, suggesting involvement of other molecular mechanisms. The results connect CK2 with apoptosis and the mechanism of thrombocytopoiesis that follow megakaryocytopoiesis. It has been previously shown that platelet shedding results from a constitutive form of apoptosis of megakaryocytes. The present investigation shows for the first time that CK2 inhibition induces release of functional platelets from malignant megakaryoblasts. In megakaryoblasts, CK2 inhibition first produces proliferation arrest, followed by differentiation to megakaryocytes that culminate with proplatelets formation, blebbing, and compartmentalized fragmentation of megakaryocytes, finalized by thrombocytes release. Platelets obtained in culture, following CK2α inhibition are functional. These platelets form a clot visible with the eye when exposed to agonists. The present invention successfully stopped the abnormal proliferation of a transformed cell line and reversed the path towards its normal function.

In conclusion, CK2α inhibition studies with TBB and DMAT, demonstrate a key role of CK2 in oncogenic development as well as in the megakaryocytopoiesis and thrombocytopoiesis processes. This opens up the possibility of CK2 targeting drug design for patients with cytokine and BCR/ABL inhibitors resistance.

### SUMMARY

In summary, MCF-7 as well as SW-480 showed proliferation arrest in the presence of 20 μM DMAT (FIGS. 16 and 33 respectively). WM-164 was more resistant to DMAT, (FIG. 50). In conclusion, all four cell lines grew forming a lot of colonies in the control experiments, in the absence of DMAT; while much smaller and fewer colonies were observed in the presence of DMAT. These combined data demonstrate that DMAT prevents proliferation of malignant cells in vitro. These data suggest that phosphorylation of a nuclear protein by CK2 is required for malignant cell proliferation.

Regarding the creation of mice xenografts (using MEG-01, MCF-7, SW-480 and WM-164 cells), the in vivo results also demonstrate that the best response to this treatment was obtained with MEG-01 and MCF-7 cells. The data obtained with these two cell lines showed tumor ablation (FIGS. 9 and 30 respectively). The data shown in FIGS. 47 and 64 with the xenografts obtained with SW-480 cells (female mice) and WM-164 cells (male mice) demonstrate significant reduction in tumor volume. However, at the end of the treatment small tumors remained (FIGS. 47 and 64). Overall, the data demonstrate that DMAT can be used as a therapy to treat tumors in vivo. Altogether the results strongly suggest that CK2 is translocated in the nucleus of malignant cells and phosphorylates a protein required for cell proliferation perturbing its normal activity.

All tumors from treated mice showed high necrotic and apoptotic areas versus untreated (DMSO) control tumors. All other organs of the DMAT treated mice appeared normal, based on hematoxylin-eosin staining. Since DMAT and TBB were solubilized in DMSO, initial control experiments demonstrate that DMSO alone has no effect on malignant cells proliferation rate and apoptosis. Very recent data demonstrated that injections of DMAT in mice (25 mg/kg twice daily) had no effect on the kidney, bone marrow, and liver of mice as assessed histologically. Similar toxicity experiments have been performed. A mouse was treated with 10 mg/day/animal for 2 weeks. Histological analyses of all organs did not show any difference between a control animal (not injected) and the mouse injected with DMAT. In conclusion, DMAT appears to be of utmost importance as a tool in developing a new cancer therapy both because of its efficacy and its apparent lack of toxicity.

A protein marker (CK2) has been identified that is translocated to the nucleus in malignant cells. Inhibition of the function of this protein by a specific inhibitor results in the arrest of cell proliferation. Therefore, phosphorylation of nuclear proteins participating in cell growth by CK2 is required for the survival of these malignant cells. Identification of the proteins responsible for abnormal cell proliferation in all these cell lines is a major contribution to the field.

Inhibition of CK2α with the inhibitors DMAT and TBB, in vitro, in MEG-01 megakaryoblastic cells, results in proliferation arrest, apoptosis, megakaryoblast differentiation to megakaryocyte and functional platelets release.

Inhibition of CK2α with the inhibitor DMAT, in vivo, in MEG-01, MCF-7, SW-480, WM-164, ACHN, HT-1376, U-87 xenograft murine models, results in proliferation arrest and tumor ablation, suggesting that small chemical compounds that inhibit kinases have strong potential in cancer treatment.

It is contemplated that the preferred inhibitor DMAT can be effectively used in a variety of cancer treatment regimes, and specifically, in the treatment of chronic myelogenous leukemia, breast cancer, colon cancer, and melanoma. In view of the significant results exhibited by the various procedures and studies described herein using DMAT, it is also contemplated that the other preferred embodiment inhibitor TBB, can also be used in corresponding treatment regimes. Furthermore, the present invention is not limited to the use of DMAT and TBB alone or in combination, but also includes the use of other CK2 inhibitors, and particularly, CK2α inhibitors.

Many other benefits will no doubt become apparent from future application and development of this technology.

All patents, patent applications, and literature cited or referenced herein, is incorporated by reference herein.

The headings used herein are merely for the convenience of the reader and shall in no way limit the scope of the present invention.

As described hereinabove, the present invention solves many problems associated with previously known approaches and treatment strategies. However, it will be appreciated that various changes in the details, materials and arrangements of parts, which have been herein described and illustrated in order to explain the nature of the invention, may be made by those skilled in the art without departing from the principle and scope of the invention, as expressed in the appended claims.

What is claimed is:

1. A method for treating a disease characterized by over-proliferation of malignant cells, the disease selected from the group consisting of (i) breast cancer, (ii) colon cancer, (iii) skin cancer, (iv) chronic myelogenous leukemia, (v) renal cell carcinoma, (vi) bladder cancer, and (vii) glioblastoma, the method comprising:
selectively inhibiting CK2α activity.

2. The method of claim 1 wherein selectively inhibiting CK2α activity comprises administering an amount of a CK2α selective inhibitor effective to arrest proliferation of the malignant cells.

3. The method of claim 2 wherein the CK2α selective inhibitor is selected from the group consisting of (i) 4,5,6,7-Tetrabromobenzotriazole (TBBt), (ii) 2-Dimethylamino-4,5,6,7-tetrabromo-1H-benzimidazole (DMAT), and combinations of (i) and (ii).

4. The method of claim 3 wherein the CK2α selective inhibitor is DMAT.

5. The method of claim 4 wherein DMAT is used in a concentration of from 0.1 μM to 1,000 μM.

6. The method of claim 5 wherein DMAT is used in a concentration of from about 1 μM to about 100 μM.

7. The method of claim 6 wherein DMAT is used in a concentration of from about 10 μM to about 50 μM.

8. The method of claim 3 wherein the CK2α selective inhibitor is TBBt.

9. The method of claim 8 wherein TBBt is used in a concentration of from 0.1 μM to 1,000 μM.

10. The method of claim 9 wherein TBBt is used in a concentration of from about 1 μM to about 150 μM.

11. The method of claim 10 wherein TBBt is used in a concentration of from about 15 μM to about 75 μM.

12. The method of claim 2 wherein the CK2α selective inhibitor is administered for a period of from about 1 to about 14 days.

13. The method of claim 12 wherein the CK2α selective inhibitor is administered for a period of from about 3 to about 7 days.

14. The method of claim 2 wherein the CK2α selective inhibitor is administered in a dosage unit of from about 0.001 to about 100 mg/kg.

15. The method of claim 14 wherein the CK2α selective inhibitor is administered in a dosage unit of from about 0.01 to about 50 mg/kg.

16. The method of claim 15 wherein the CK2α selective inhibitor is administered in a dosage unit of from about 0.05 to about 20 mg/kg.

17. A method for treating a disease characterized by over-proliferation of malignant cells, the disease selected from the group consisting of (i) breast cancer, (ii) colon cancer, (iii) skin cancer, (iv) chronic myelogenous leukemia, (v) renal cell carcinoma, (vi) bladder cancer, and (vii) glioblastoma the method comprising:
administering an effective amount of a CK2α inhibitor to a patient in need of treatment.

18. The method of claim 17 wherein selectively inhibiting CK2α activity comprises administering an amount of a CK2α selective inhibitor effective to arrest proliferation of the malignant cells.

19. The method of claim 18 wherein the CK2α selective inhibitor is selected from the group consisting of (i) 4,5,6,7-Tetrabromobenzotriazole (TBBt), (ii) 2-Dimethylamino-4,5,6,7-tetrabromo-1H-benzimidazole (DMAT), and combinations of (i) and (ii).

20. The method of claim 19 wherein the CK2α selective inhibitor is DMAT.

21. The method of claim 20 wherein DMAT is used in a concentration of from 0.1 μM to 1,000 μM.

22. The method of claim 21 wherein DMAT is used in a concentration of from about 1 μM to about 100 μM.

23. The method of claim 22 wherein DMAT is used in a concentration of from about 10 μM to about 50 μM.

24. The method of claim 19 wherein the CK2α selective inhibitor is TBBt.

25. The method of claim 24 wherein TBBt is used in a concentration of from 0.1 μM to 1,000 μM.

26. The method of claim 25 wherein TBBt is used in a concentration of from about 1 μM to about 150 μM.

27. The method of claim 26 wherein TBBt is used in a concentration of from about 15 μM to about 75 μM.

28. The method of claim 18 wherein the CK2α selective inhibitor is administered for a period of from about 1 to about 14 days.

29. The method of claim 28 wherein the CK2α selective inhibitor is administered for a period of from about 3 to about 7 days.

30. The method of claim 18 wherein the CK2α selective inhibitor is administered in a dosage unit of from about 0.001 to about 100 mg/kg.

31. The method of claim 30 wherein the CK2α selective inhibitor is administered in a dosage unit of from about 0.01 to about 50 mg/kg.

32. The method of claim 31 wherein the CK2α selective inhibitor is administered in a dosage unit of from about 0.05 to about 20 mg/kg.

33. A pharmaceutical composition comprising:
a CK2α selective inhibitor selected from the group consisting of (i) 4,5,6,7-Tetrabromobenzotriazole (TBBt), (ii) 2-Dimethylamino-4,5,6,7-tetrabromo-1H-benzimidazole (DMAT), and combinations of (i) and (ii); and
a pharmaceutically acceptable carrier.

34. The composition of claim 33 wherein the CK2α selective inhibitor is TBBt.

35. The composition of claim 33 wherein the CK2α selective inhibitor is DMAT.
